(19) United States
(12) Patent Application Publication
(10) Pub. No.: US 2026/0023036 A1
(43) Pub. Date: Jan. 22, 2026

(54) Methods and Systems for Determining Soil Texture Using Mobile Gamma Analysis

(71) Applicants: The United States of America, as represented by the Secretary of Agriculture, Washington, DC (US); Carbon Asset Solutions (USA), Inc., Wetumpka, AL (US)

(72) Inventors: Daniel Donner, Winnipeg (CA); Henry Allen Torbert, III, Opelika, AL (US); Galina N. Yakubova, Auburn, AL (US); Aleksandr G. Kavetskiy, Auburn, AL (US); Stephen A. Prior, Auburn, AL (US)

(73) Assignees: The United States of America, as represented by the Secretary of Agriculture, Washington, DC (US); Carbon Asset Solutions (USA), Inc., Wetumpka, AL (US)

(21) Appl. No.: 19/272,951

(22) Filed: Jul. 17, 2025

Related U.S. Application Data
(60) Provisional application No. 63/673,590, filed on Jul. 19, 2024.

(51) Int. Cl. G01N 23/222; G01N 33/24; G21F 1/08; G21F 1/10

(52) U.S. Cl. CPC G01N 23/222; G01N 33/245; G21F 1/085; G21F 1/106; G01N 2223/0745; G01N 2223/1013; G01N 2223/1045; G01N 2223/1063; G01N 2223/1066; G01N 2223/3303; G01N 2223/616

(57) ABSTRACT

A method for identifying a soil texture class of a soil using gamma analysis comprises: acquiring an inelastic neutron scattering (INS) gamma spectrum of the soil; calculating at least one ratio of a mass fraction of a first oxide to a mass fraction of a second oxide present in the soil, wherein the mass fraction of each of the first and second oxides is determined from calculating a contribution to a characteristic peak in the gamma spectrum of the soil by each oxide of the first and second oxides; and identifying one or more soil texture classes of the soil by identifying a contour line of a contour plot that corresponds to the calculated at least one ratio, wherein the contour line correlates the calculated at least one ratio to one or more soil texture classes. A mobile system for gamma analysis determination of soil texture is also provided.

### CROSS REFERENCE TO RELATED APPLICATIONS

This application claims the benefit of U.S. Provisional Application No. 63/673,590 filed on Jul. 19, 2024 and entitled “Methods and Systems for Determining Soil Texture Using Mobile Gamma Analysis,” all of which is incorporated herein by reference.

### TECHNICAL FIELD

The present disclosure relates to systems and methods for identifying the soil texture of a soil, in particular, by using gamma analysis.

### BACKGROUND

Soil texture is a function of the physical size of the mineral fraction particles that are present in a soil. Soil scientists have established three particle size range components for particles that are present in soil; namely, sand, silt and clay. These particles size components are defined as follows:
Sand—0.05 to 2.00 mm
Silt—0.002 to 0.05 mm
Clay—less than 0.002 mm

Soil texture is defined by the relative percentage of sand, silt and clay contained in a soil. Using the relative percentage of these three particle size components, soil scientists have defined twelve soil texture classes. By determining the percentage of each of these particle size components present in any soil, the soil texture classification of that soil may be identified as set out in the triangle diagram of FIG. 1. In general, the twelve soil texture classes are separated by relatively large shifts in the percentage content of sand, silt and clay. However, the amount of clay in the soil tends to dominate the soil texture classification.

Of the numerous minerals found in sand, clay, and silt, the chemical composition of soil minerals may be represented by a set of oxides, with the primary ones being SiO₂, Al₂O₃, Fe₂O₃, CaO, and MgO. Soil composition can often be represented as set of these oxides along with elemental carbon (C) and water.

The soil texture classification of a soil is a characteristic of the soil that affects how the soil functions and how the soil needs to be managed to optimize the growth of crops. The soil texture of a soil impacts the water-holding capacity and permeability of the soil, and the capacity of the soil to retain nutrients. The appropriate application rates of fertilizers, herbicides, water and other inputs may change depending on the soil texture classification of the soil.

Soil texture is an essential soil characteristic that drives soil functions and plant productivity, and therefore plays an important role in determining how cropping systems should be managed. Existing models use soil texture as an input into their algorithms for determining soil functions. While models have been shown to be accurate when the correct baseline data is entered, there is a direct correlation between the predictive capacity of the model and correctly inputting the soil texture of the soil to be analyzed. Existing national soil databases may provide georeferenced soil texture information, but models using such soil texture inputs as an approximation of the soil texture for a particular field have been demonstrated to produce widely different predictions for changes in management practices in prediction water quality, when using different databases to approximate the soil texture of the same field as an input into the models (Prasanna H. Gowda, P. H., and D. J. Mulla. 2005. Watershed Management to Meet Water Quality Standards and Emerging TMDL (Total Maximum Daily Load). Proceedings of the Third Conference 5-9 Mar. 2005. ASAE Publication Number 701P0105, ed. P. W. Gassman, (Atlanta, Georgia USA).

Likewise, soil texture is the main soil characteristic that determines soil water holding capacity. Therefore, soil texture is an important soil characteristic for managing irrigation. The USDA has developed a Variable-Rate Irrigation Decision Support System (VRIDSS) to be used for determining the best time and rate of irrigation to be used in production agriculture (Stone, K. C., P. J. Bauer, S. O'Shaughnessy, A. Andrade-Rodriguez, and S. Evett. 2020. A variable-rate irrigation decision support system for corn in the U.S. eastern coastal plain. ASABE Vol. 63 (5): 1295-1303 ISSN 2151-0032 https://doi.org/10.13031/trans.13965). The VRIDSS tool may be used for precision application of irrigation across a field, but research has shown that accounting for changes in soil texture, especially sand content, across a field is essential for this tool to work accurately (Vories, E., S. O'Shaughnessy, K. Sudduth, S. Evett, M. Andrade, and S. Drummond. 2021. Comparison of precision and conventional irrigation management of cotton and impact of soil texture. Precision Agriculture 22:414-431. https://doi.org/10.1007/s11119-020-09741-3).

Thus, accurate identification of the soil texture of a soil, which varies across a field, may be important information for precision farming techniques. However, existing methods for determining soil texture involve field sampling and soil analysis, which is time consuming, labor-intensive, and may be costly as a result. Furthermore, basing soil texture determination on field sampling introduces the possibility that significant changes in soil texture in a given location may be missed, if the locations from which the samples were taken do not include the area of the field where significant soil texture changes occur. There is a need for improved methods of soil texture determination, and for identifying and mapping changes in soil texture across a field.

### SUMMARY

In one aspect of the present disclosure, the applicants have discovered that by utilizing mobile gamma analysis to determine the ratios of particular elements or mineral compounds in a soil, this information may be used to identify the soil texture class of the soil under analysis. For example, sand and silt are primarily made up of quartz, which is a crystalline framework of silicon-oxygen tetrahedra having a chemical formula of SiO₂. Therefore, soil textures that result from a higher percentage of sand and silt will also have a higher percentage of Si. Additionally, while both silt and sand are made primarily of SiO₂, the applicant has noted that because the particle size of silt is dramatically smaller than the particle size of sand, the density of Si within a given volume of soil would be much higher for a soil with a larger silt component, as compared to a soil with a larger sand component. Thus, although there would be little difference between sand and silt for the concentration of Si based on gravimetric measurements, there would be significant differences in the concentration of Si based on volumetric measurements.

Clay minerals that make up the clay portion of a soil are more complex, generally consisting of sheet layers of SiO₄ tetrahedra and AlO₄ octahedra structures. As a result, clay minerals contain somewhere between 20% to 40% Al₂O₃. Therefore, soil textures containing a higher percentage of clay should also have a relatively higher percentage of Al. In one aspect of the present disclosure, performing a mobile gamma analysis on a soil to calculate a ratio of Al₂O₃:SiO₂ contained within that soil may be used to identify the soil texture classification of the soil under analysis. In some cases, the ratio of Al₂O₃:SiO₂ may not, alone, allow for the determination of the exact soil texture classification of the soil. However, this ratio may be used to identify a limited number of soil texture classes that the soil under measurement belongs to, which information may be used, for example, to determine whether the soil is clay-dominant or sand and silt-dominant. Thus, an approximate identification of the soil texture class of a soil may still be used to guide decisions on farming practices, as small changes in the distribution percentages of sand, silt and clay of a given soil may not be significant enough to warrant changes in farming practices.

In another aspect of the present disclosure, additional soil element ratios may be measured to assist in further identifying the class of soil texture of a soil under analysis. For example, the use of other soil element ratios to distinguish changes in soil have been used by geologists to develop indexes for measuring weathering of a soil; as a general rule, the more weathered a soil is, the more clay it contains. The applicants hypothesize that measuring the ratios of different elements contained in a soil, as determined by gamma analysis, may result in defining a correlative relationship between those ratios and the soil texture classes, as the applicants have shown for the ratio of Al₂O₃:SiO₂ of a soil. For example, based on existing data regarding the relative content of the oxide Fe₂O₃ in certain minerals that are commonly found in soils, ratios of Fe₂O₃ with SiO₂ and/or Al₂O₃, may prove to be additionally useful for determining soil content through mobile gamma analysis. However, this is not intended to be limiting, and it will be appreciated by a person skilled in the art that ratios of other elements, oxides, or other compounds that are found in soil, which are capable of measurement by gamma analysis, may also be used to determine soil texture in accordance with the present disclosure. In one aspect, the applicants hypothesize that combining two or more different elemental ratios of a soil, as measured by mobile gamma analysis, may be used to further refine the identification of the soil texture of a soil under analysis with greater precision.

In one aspect of the present disclosure, a method for identifying a soil texture class of a soil is provided. In some embodiments, the method comprises the following steps: acquiring an inelastic neutron scattering (INS) gamma spectrum of the soil; calculating at least one ratio of a mass fraction of a first oxide to a mass fraction of a second oxide present in the soil, wherein the mass fraction of each of the first and second oxides is determined from calculating a contribution to a characteristic peak in the gamma spectrum of the soil by each oxide of the first and second oxides; and identifying one or more soil texture classes of the soil by identifying a contour line of a contour plot that corresponds to the calculated at least one ratio, wherein the contour line correlates the calculated at least one ratio to one or more soil texture classes.

In some embodiments, the step of calculating the at least one ratio of the mass fractions of the first and second oxides further comprises performing a deconvolution procedure on the acquired gamma spectrum, wherein the deconvolution procedure applies a least squares method for determining the mass fraction of each of the first and second oxides. In some embodiments, the deconvolution procedure is modified to account for radiation attenuation by components in the soil. In some embodiments, the first oxide may be SiO₂. In some embodiments, the second oxide may be selected from a group comprising: Al₂O₃, Fe₂O₃.

In some embodiments of the method, the INS gamma spectrum of the soil may be acquired using a Tagged Neutron Method (TNM) system. In some embodiments, the soil is in a field and the step of acquiring the INS gamma spectrum of the soil further comprises moving the TNM system across the field in a point sampling mode to obtain a plurality of INS gamma spectra of the soil.

In some embodiments of the method, the INS gamma spectrum of the soil may be obtained using a Pulsed Fast Thermal Neutron Analysis (PFTNA) system. In some embodiments, the soil is in a field and the step of acquiring the INS gamma spectrum of the soil further comprises moving the PFTNA system across the field in a scanning mode to obtain a plurality of INS gamma spectra of the soil.

In some embodiments, the method further includes the step of acquiring geographic coordinates for each position on the field where each INS gamma spectrum of the plurality of INS gamma spectra is obtained.

In some embodiments, the step of calculating the at least one ratio of the mass fraction of the first oxide to the mass fraction of the second oxide present in the soil comprises calculating a first ratio of the mass fraction of the first oxide to the mass fraction of the second oxide present in the soil, and calculating a second ratio of a mass fraction of a third oxide to a mass fraction of a fourth oxide present in the soil, and the step of identifying one or more soil texture classes of the soil comprises: identifying a contour line of a first contour plot that corresponds to the calculated first ratio, the identified contour line of the first contour plot correlating the first calculated ratio to a first grouping of one or more soil texture classes; identifying a contour line of a second contour plot that corresponds to the calculated second ratio, the identified contour line of the second contour plot correlating the second calculated ratio to a second grouping of one or more soil texture classes; identifying an overlap between the first and second groupings of one or more soil texture classes to determine the soil texture class of the soil. In some embodiments, the second and fourth oxides may both be SiO₂.

In another aspect of the present disclosure, a system for identifying a soil texture class of a soil is provided. In some embodiments, the system comprises: a neutron generator assembly for generating neutrons and directing the generated neutrons into the soil; a gamma detector assembly for detecting the gamma radiation emitted by the soil; a radiation shielding positioned between the neutron generator assembly and the gamma detector assembly; a processor in communication with the gamma detector assembly. The processor may be configured to: acquire an INS gamma spectrum from the gamma radiation detected by the gamma detector assembly; calculate a mass fraction of each of at least a first and second oxide present in the soil, each mass fraction of each oxide based on a net peak area of a characteristic peak of each oxide obtained from the acquired gamma spectrum; calculate at least one ratio of the mass fractions of the at least first and second oxides present in the soil; and record the acquired gamma spectrum and the calculated at least one ratio to a memory.

In some embodiments of the system, the neutron generator assembly also generates alpha particles and the system further comprises an alpha detector assembly. In some embodiments, the gamma detector assembly may be positioned spaced apart from, and laterally of, the neutron generator. In some embodiments, the alpha detector assembly and the soil are positioned on opposite sides of the neutron generator assembly. In some embodiments, the radiation shielding comprises one or more of the following: lead, borated polyethylene, borated-lead polyethylene.

In some embodiments, the system may be mounted to a mobile cart and the system further comprises a global positioning system (GPS). In such embodiments, the processor may be configured to record a plurality of gamma spectra of the soil and to save a geographic coordinate of the location of each acquired gamma spectrum of the plurality of gamma spectra to the memory.

In some embodiments, the processor is additionally configured to identify one or more soil texture classes of the soil by identifying a contour line of a contour plot that corresponds to the calculated at least one ratio, wherein the contour plot correlates the calculated at least one ratio to one or more soil texture classes.

In some embodiments, the processor is additionally configured to perform a deconvolution procedure on the acquired gamma spectrum, the deconvolution procedure comprising applying a least squares method for determining the mass faction of each of the at least first and second oxides present in the soil. In some embodiments, the deconvolution procedure may be modified to account for radiation attenuation by components in the soil.

In another aspect of the present disclosure, the applicants hypothesize that measurements of the natural soil background gamma radiation, produced by radioactive elements naturally present in the soil, may be used to estimate or identify the soil texture classification of a soil. For example, not intended to be limiting, soil may contain one or more of the following primordial radioisotopes:
Potassium: ⁴⁰K decays to produce ⁴⁰Ar
Thorium: ²³²Th decays to produce ²⁰⁸Tl
Uranium: ²³⁸U decays to produce ²¹⁴Bi

The gamma radiation produced by the decay of Potassium-40, Thallium-208 and Bismuth-214 produces characteristic gamma peaks. The different mineral components of the silt, sand and clay of a given soil will contain different amounts of the above-noted primordial radioisotopes. Therefore, the applicant hypothesizes that measurement of the natural background gamma radiation of a soil, using mobile gamma analysis, may be used to correlate such measurements to different soil texture classes. In one example, this may involve correlating the total counts in the gamma spectrum, representing the natural gamma background radiation in the soil, to varying content of sand, silt and clay contained in the soil. In another example, a measurement of the volumetric concentration of one or more of the primordial radioisotopes, mentioned above, may be correlated with the percentage of silt, sand, and/or clay in a soil under analysis.

In some embodiments, the measurement of the natural soil background gamma radiation may be utilized, alone, to determine an approximate soil texture classification. Whereas, in other embodiments, a combination of the measurement of the natural soil background gamma radiation and measurements of one or more oxide ratios, as determined by inelastic neutron scattering measurements, may be used to determine an approximate soil texture classification of the soil under analysis.

### BRIEF DESCRIPTION OF THE DRAWINGS

FIG. 1 is a triangle diagram illustrating the soil texture classes, as is known in the prior art.

FIG. 2 is a 3D plot illustrating the relationship between the ratio of the oxides Al₂O₃:SiO₂ of a soil to the clay and sand content of the soil.

FIG. 3 is a contour plot of the ratio of the oxides Al₂O₃:SiO₂ of a soil, as shown in FIG. 2, projected onto the sand-clay content plane of the plot of FIG. 2, with the contour lines indicating the calculated Al₂O₃:SiO₂ ratio values.

FIG. 4 is a 2D representation of the soil texture class triangle diagram of FIG. 1, showing only the sand and clay components of a soil.

FIG. 5 is the contour plot of FIG. 3 overlaid onto the 2D representation of the soil texture class diagram of FIG. 4.

FIG. 6 is a schematic diagram, showing the geometrical relationship and relative positioning between the components of an embodiment of a Tagged Neutron Method (TNM) system for acquiring gamma spectra from a soil.

FIG. 7 is a schematic diagram, showing the electrical components of the embodiment of the TNM system of FIG. 6.

FIG. 8A is a screenshot obtained from the IGOR™ software application, displaying an example of an acquired alpha pulse obtained from a TNM system.

FIG. 8B is a screenshot obtained from the IGOR™ software application, displaying an example of an acquired gamma pulse obtained from a TNM system.

FIG. 9 is an example of a time-of-flight (TOF) spectrum generated by the IGOR software application, the TOF spectrum obtained from a TNM system.

FIG. 10 is a graph displaying the simulated gamma spectra of oxides and water in a soil, and of the soil itself, plotted against a varying volume of a simulated cylindrical sample of the soil, the cylindrical sample having a radii of f and a thickness (or height) of f measured in centimeters.

FIG. 11 is a schematic diagram, providing an example of a TNM system configuration and the related measurements and inputs into the equations for converting the acquired gamma spectra to account for radiation attenuation.

FIG. 12 is an example of the gamma spectra measured by a TNM system using reference samples of individual oxides, carbon and water and of Soil Bin 3a, each sample having a volume of approximately 0.5 m³.

FIG. 13 is an example of the gamma spectrum obtained of the soil sample in Soil Bin 3a after performing the deconvolution process, to separate out the contributions to the gamma spectrum of the soil sample by each of the individual components found within the soil. The difference between the spectrum generated from summing the spectra obtained for each of the individual soil components through the deconvolution process, and the original gamma spectrum obtained from the soil sample in Bin 3a, is shown at the top of the graph.

FIG. 14 is an example of a background gamma spectrum measured for the top 30 cm of a sample of soil, showing the background radiation produced by radioactive elements that are naturally present in a soil.

FIG. 15A is a plot showing the relationship between total counts (TC) measured to a depth of 10 cm in a background gamma spectra of a soil sample and the % sand content of the soil sample.

FIG. 15B is a plot showing the relationship between total counts (TC) measured to a depth of 10 cm in a background gamma spectra of a soil sample and the % silt content of the soil sample.

FIG. 15C is a plot showing the relationship between total counts (TC) measured to a depth of 10 cm in a background gamma spectra of a soil sample and the % clay content of the soil sample.

FIG. 16 is a plot showing the relationship between the logarithmic measured content of Thorium (ppm) and the logarithmic percentage of clay in a soil sample.

FIG. 17 is a Bland-Altman plot of the moisture measurements performed by TNM and by time domain reflectometry (TDR) on soils.

FIG. 18 is a Deming regression line plot (represented by the solid line) performed on the data of moisture measurements performed by TNM and by TDR on soils (represented by the points with the vertical and horizontal error bars, respectively).

FIG. 19 is a Bland-Altman plot of the carbon content measurements performed by TNM and by dry combustion on soils.

FIG. 20 is a Deming regression line plot (represented by the solid line) performed on the carbon content measurements performed by TNM and by dry combustion on soils (represented by the points with the vertical and horizontal error bars, respectively).

FIG. 21 is a Bland-Altman plot of the calculated

$$\frac{Soil {Al}_{2} {O}_{3}}{Soil {SiO}_{2}}$$

ratio of different soils, each ratio of each soil calculated from both a reference source and from TNM measurement data.

FIG. 22 is a Deming regression line plot (represented by the solid line) performed on the calculated

$$\frac{Soil {Al}_{2} {O}_{3}}{Soil {SiO}_{2}}$$

ratio of different soils, each ratio of each soil calculated from both a reference source and from TNM measurement data (represented by the points with the horizontal and vertical reference bars, respectively).

### DETAILED DESCRIPTION

In one aspect of the present disclosure, the approximate classification of the soil texture of a soil may be identified, based on the measurement of the relative ratios of particular compounds in the soil as determined by mobile gamma analysis. In an illustrative embodiment, as described herein, the approximate soil texture class of a soil may be determined by measuring the ratio of Al₂O₃:SiO₂ in a soil and correlating this measured ratio to different soil texture classifications, as previously determined from a plurality of other measurements of Al₂O₃:SiO₂ in different soil samples. Although an illustrative example will be provided with reference to measurements of the ratio of Al₂O₃:SiO₂ in a soil, it will be appreciated by a person skilled in the art that the correlation of other soil element ratios to the soil texture classes may also be used in the approximate classification of the soil texture of a soil. In some embodiments, the measurement of two or more ratios of different soil elements of a soil may be used to more precisely determine the soil texture class of a soil.

It will be appreciated that it may not be necessary to obtain a precise soil texture classification in order to obtain useful information for guiding farming practices. For example, small changes in the percentage of distribution in a soil as between sand, silt and clay may not be important for optimizing farming practices; however, the difference between a sand-dominant soil texture and a clay-dominant soil texture may be usefully detected by the methods and apparatuses described herein, and such information may be used for optimizing farming practices.

As an example of how farming practices may be optimized based on the soil texture classification of a soil, not intended to be limiting, the application rates specified on the label for many premerger herbicides varies based on the level of clay in soil. The general clay content level in a soil may be all that is required to comply with herbicide application regulations, rather than based on a precise determination of the clay content level in the soil. However, improved herbicide efficiency and reduced costs may be achieved by utilizing precision herbicide application, based on a determination of the changes of soil clay content across a field.

### Correlating Soil Elemental Ratios and Soil Textures

Soil texture is a function of the physical particle sizes that are present in any soil. The particle size components of soil are: sand (0.05 to 2.00 mm); Silt (0.002 to 0.05 mm); and clay (less than 0.002 mm). The challenge of determining the soil texture classification of a soil, based on an analysis of the elemental content of the soil, is that each particle size component may be made up of different minerals having different chemical compositions, with the elements of silicon and aluminum comprising a large percentage of the elements found in sand, silt and clay. One possible method of differentiation between sand, silt and clay is to determine the volumetric concentration of each element in a soil sample, on the basis that the volumetric concentration of an element in a coarser soil component (such as the concentration of silicon in sand) should be much less than the volumetric concentration of the same element in a finer soil component (such as the concentration of silicon in silt).

Regarding the elemental content of each soil texture component, an approximation of the elemental content of each soil texture component is provided in Table 1, below. While the chemical composition of the soil components may vary, the primary oxides found in a soil are SiO₂, Al₂O₃, Fe₂O₃, CaO, and MgO, with the remainder of the soil being comprised of carbon and water. It will be appreciated that each soil mineral component is represented as a set of oxides, consisting primarily of silicon dioxide (silica, SiO₂) and aluminum oxide (alumina, Al₂O₃), as reflected in Table 1.

TABLE 1
Main soil minerals in sand, clay, and silt along with chemical formula and oxide content

| Soil | | | Main Oxide component | | |
|---|---|---|---|---|---|
| compo- | | | Oxide | | |
| nent | Mineral | Chemical Formula | formula | Content, % | Reference |
| Sand | Silica | SiO₂, Al₂O₃, Fe₂O₃, | SiO₂ | ~97 | Katsina, 2013 |
| | | CaO . . . | Al₂O₃ | ~2 | BSG Glass Chip, 2024 |
| | | | Other | ~1 | |
| Clay | Kaolinite | Al₄Si₄O₁₀(OH)₈ | SiO₂ | 46.5 | Murray, 2006 |
| | | | Al₂O₃ | 39.5 | |
| | | | H₂O | 14.0 | |
| | Smectite | (OH)₄Si₈Al₄O₂₀•nH₂O | SiO₂ | 66.7 | Murray, 2006 |
| | | | Al₂O₃ | 28.3 | |
| | | | H₂O | 5 | |
| | Montmorillonite | (Na, Ca)_{0.33}(Al, | SiO₂ | 43.5 | Mineralogy Database, |
| | | Mg)₂(Si₄O₁₀) | Al₂O₃ | 18.4 | 2014a |
| | | (OH)₂•nH₂O | CaO | 1.0 | |
| | | | Na₂O | 1.1 | |
| | | | H₂O | 36.0 | |
| Silt | Quartz | SiO₂ | SiO₂ | 100 | Mineralogy Database, |
| | | | | | 2014b |
| | Kaolinite | Al₄Si₄O₁₀(OH)₈ | SiO₂ | 46.5 | Murray, 2006 |
| | | | Al₂O₃ | 39.5 | |
| | | | H₂O | 14.0 | |
| | Chlorite | (OH)₄(Si Al)₈(Mg—Fe)₆O₂₀ | SiO₂ | 25 | Gailhanou et al., 2009 |
| | | | Al₂O₃ | 20 | |
| | | | FeO | 19.4 | |
| | | | Fe₂O₃ | 2.7 | |
| | | | MgO | 18.8 | |
| | | | H₂O | 11.9 | |
| | | | Other | 2.2 | |
| | Mica | XY₂₋₃Z₄O₁₀(OH)₂ | SiO₂ | 46.4 | Prasada et al., 2013 |
| | | X = K, Na or Ca | Al₂O₃ | 36.8 | |
| | | Y = Al, Mg, Fe . . . | H₂O | 3.2 | |
| | | Z = Si, Al | Other | 13.6 | |
| | Smectite | (OH)₄Si₈Al₄O₂₀•nH₂O | SiO₂ | 66.7 | Murray, 2006 |
| | | | Al₂O₃ | 28.3 | |
| | | | H₂O | 5 | |
| | Feldspars | KAlSi₃O₈ | SiO₂ | 68 | Othman et al., 2017 |
| | | NaAlSi₃O₈ | Al₂O₃ | 22 | |
| | | CaAl₂Si₂O₈ | K₂O | 3 | |
| | | | Other | 7 | |

### References for Table 1

1. BSG Glass Chip, 2024. Understanding silica sand: Composition & characteristics. Available at: https://bsgglasschip.com/understanding-silica-sand/2.
2. Katsina, C., Bala, C. K., Reyazul, H., Khan, R. H., 2013. Characterization of beach/river sand for foundry application. Leonardo J. Sci. 23, 77-83.
3. Mineralogy Database, 2012a. Montmorillonite mineral data. Available at: https://webmineral.com/data/Montmorillonite.shtml (accessed 14 Nov. 2024).
4. Murray, H. H., 2006. Development in Clay Science. Chapter 2. Structure and Composition of the Clay Minerals and their Physical and Chemical Properties. Volume 2, pp: 7-31. https://doi.org/10.1016/S1572-4352(06)02002-2
5. Gailhanou, H. et al., 2009. Thermodynamic properties of chlorite CCa-2. Heat capacities, heat contents and entropies. Geochimica et Cosmochimica Acta 73:4738-4749. doi:10.1016/j.gca.2009.04.040. https://www.researchgate.net/figure/Chemical-composition-wt-of-the-chlorite-CCa-2-sample_tbl1_248432854
6. Prasada, B. G., Paramageetham, C., Basha, S., 2013. New Facultative Alkaliphilic, Potassium Solubilizing, Bacillus Sp. SVUNM9 Isolated from Mica Cores of Nellore District, Andhra Pradesh, India. Research and Reviews: Journal of Microbiology and Biotechnology. Vol. 2 (1): 1-7. ISSN: 2320-3528.
7. R. Othman, Z. Mustafa, and L. Ting. 2017. Effects of mechanical activation on the fluxing properties of Gua Musang Feldspar. Journal of Mechanical Engineering and Sciences 11 (4): 3189-3196. DOI: https://doi.org/10.15282/jmes.11.4.2017.21.0287. ISSN (Print): 2289-4659; e-ISSN. 2231-8380

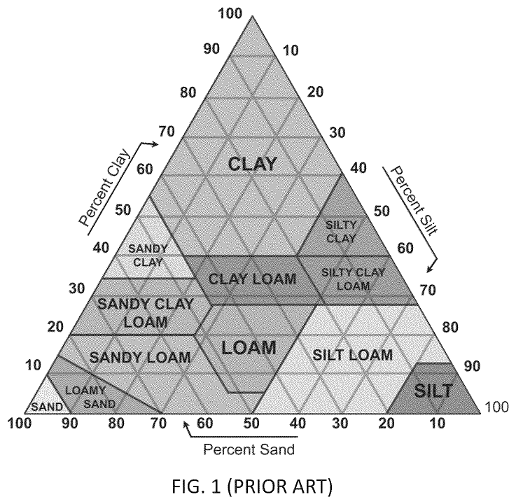

Soil texture is defined by the relative percentage of sand, silt and clay present in soil. Using the relative percentages of these three components, soil scientists have identified soil texture classes as shown in the triangle diagram of FIG. 1. To draw a correlation between the elemental content of soil and the soil texture classes shown in FIG. 1, the ratio of soil Al₂O₃ to soil SiO₂ may be determined for many different examples of soils from different fields having different soil textures. These examples were taken from information presented in the Soil Survey Geographic Database, SSURGO (Soil Survey Staff, 2024). The SSURGO database is a publicly available dataset for soils across the US provided by the USDA-NRCS. Along with soil texture, the percentages of sand, clay, and silt can be found in this database.

The knowledge of mineral content in each soil component and the oxide composition of different minerals allows for the calculation of Al₂O₃ and SiO₂ contents in each soil example, and their corresponding ratio of Soil_Al₂O₃ to Soil_SiO₂ may be calculated. Although there is variation in mineral content in each soil component, this calculation of the ratio Soil_Al₂O₃ to Soil_SiO₂ may be done for different examples of soils having varying mineral contents. The following equations were used for the calculations:

$${Sand\_SiO}_{2} = {SilicaSiO}_{2} ;$$

$${Clay\_SiO}_{2} = KaoliniteC \times {Kaolinite SiO}_{2} + SmectiteC \times {SmectiteSiO}_{2} ;$$

$${Clay\_Al}_{2} {O}_{3} = KaoliniteC \times {KaoliniteAl}_{2} {O}_{3} + SmectiteC \times {SmectiteAl}_{2} {O}_{3} ;$$

$${Silt\_SiO}_{2} = QuartzS \times {QuartzSiO}_{2} + KaoliniteS \times {Kaolinite SiO}_{2} + ChloriteS \times {ChloriteSiO}_{2} + MicaS \times {MicaSiO}_{2} + SmectiteS \times {Smectit eSiO}_{2} + FeldsparS \times {FeldsparSiO}_{2} ;$$

$${Silt\_Al}_{2} {O}_{3} = KaoliniteS \times {Kaolinite Al}_{2} {O}_{3} + ChloriteS \times {ChloriteAl}_{2} {O}_{3} + MicaS \times {MicaAl}_{2} {O}_{3} + SmectiteS \times {SmectiteAl}_{2} {O}_{3} + FeldsparS \times {FeldsparAl}_{2} {O}_{3} ;$$

$${Soil\_SiO}_{2} = X \times {Sand\_SiO}_{2} + Y \times {Clay\_SiO}_{2} + Z \times {Silt\_SiO}_{2} ; and$$

$${Soil\_Al}_{2} {O}_{3} = X \times {Sand\_Al}_{2} {O}_{3} + Y \times {Clay\_Al}_{2} {O}_{3} + Z \times {Silt\_Al}_{2} {O}_{3}$$

where:
Sand_SiO₂, Clay_SiO₂, and Silt_SiO₂ are contents of SiO₂ in sand, clay, and silt, respectively;
Clay_Al₂O₃ and Silt_Al₂O₃ are contents of Al₂O₃ in clay and silt, respectively (and assuming there is no Al₂O₃ in sand);
SilicaSiO₂ is SiO₂ content in sand (in other words, it is assumed that sand is substantially comprised of silica, for the purpose of these calculations);
KaoliniteC, SmectiteC are contents of Kaolinite and Smectite in clay, respectively;
QuartzS, KaoliniteS, ChloriteS, MicaS, SmectiteS, FeldsparS are contents of Quartz, Kaolinite, Chlorite, Mica, Smectite, and Feldspar in silt, respectively;
KaoliniteSiO₂, SmectiteSiO₂, ChloriteSiO₂, MicaSiO₂, FeldsparSiO₂, KaoliniteAl₂O₃, ChloriteAl₂O₃, MicaAl₂O₃, SmectiteAl₂O₃, FeldsparAl₂O₃ are content of SiO₂ and Al₂O₃ in these minerals (based on the data provided in Table 1);
SoilSiO₂ and SoilAl₂O₃ are content of SiO₂ and Al₂O₃ in soil, respectively;
X, Y, Z represent the content of sand, clay and silt in soil, respectively.

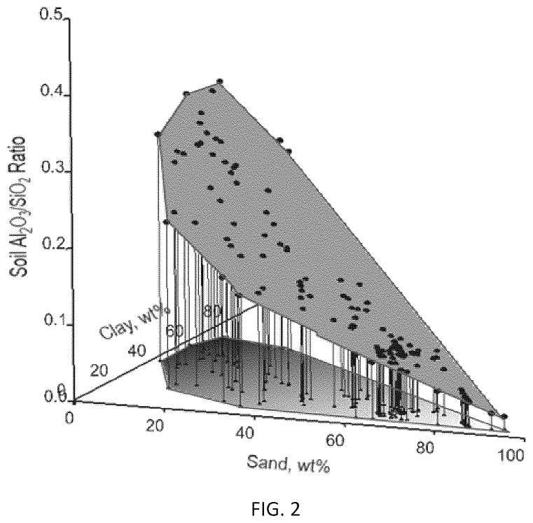

After performing the above-described ratio calculations, a 3D plot may be generated showing the calculated ratio data versus sand and clay content (FIG. 2). As shown in FIG. 2, values of the

$$\frac{Soil {Al}_{2} {O}_{3}}{Soil {SiO}_{2}}$$

ratio lie very close to the plane surface and this ratio increases with decreasing sand content. A contour plot of the dependence of the

$$\frac{Soil {Al}_{2} {O}_{3}}{Soil {SiO}_{2}}$$

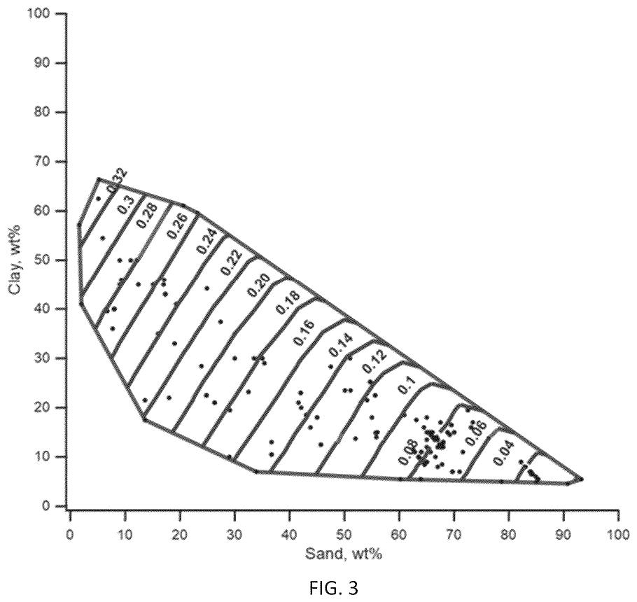

ratio on the sand-clay content plane may also be generated, as shown in FIG. 3. The contour plot shown in FIG. 3 demonstrates point positions, in sand-clay content coordinates, for which the

$$\frac{Soil {Al}_{2} {O}_{3}}{Soil {SiO}_{2}}$$

ratio was calculated. The position of the contour lines in the plot each represent the constant values of the calculated

$$\frac{Soil {Al}_{2} {O}_{3}}{Soil {SiO}_{2}}$$

ratio.

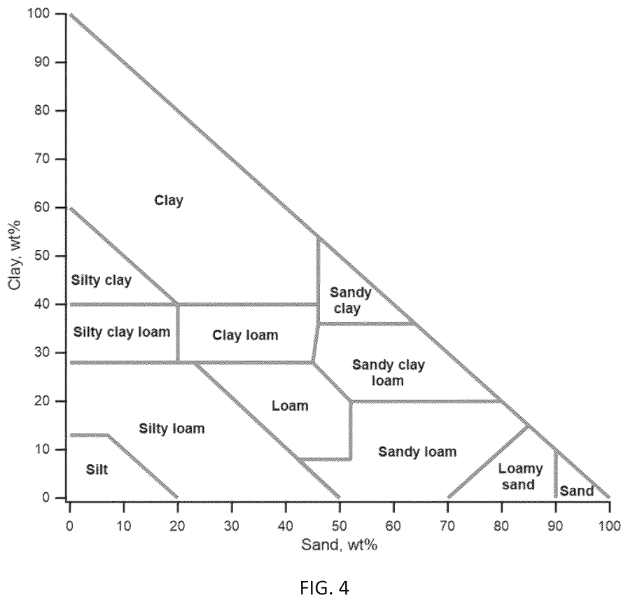

Since the percentage of the three soil texture components must add up to 100%, a transformation of the triangular soil texture diagram shown in FIG. 1 to the sand-clay plane is possible, resulting in the two-dimensional soil texture diagram shown in FIG. 4. The overlay of the contour plot of

$$\frac{Soil {Al}_{2} {O}_{3}}{Soil {SiO}_{2}}$$

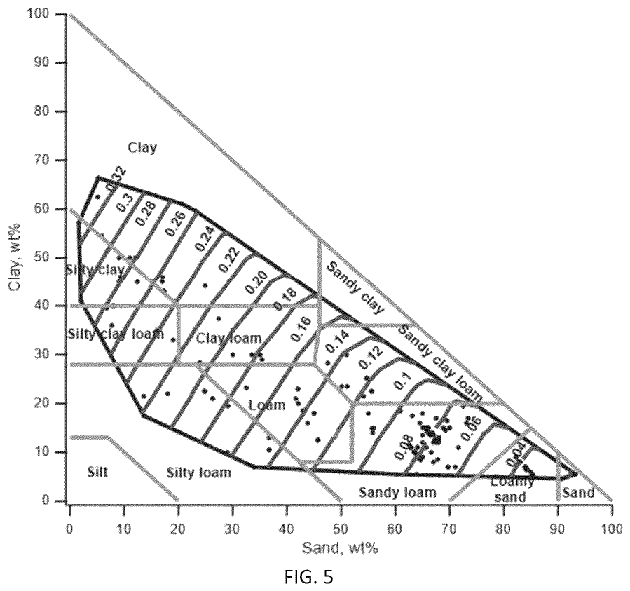

ratio versus sand and clay soil content (as shown in FIG. 3) onto the two-dimensional sand-clay plane soil texture diagram (as shown in FIG. 4) is shown in FIG. 5. From this diagram in FIG. 5, some conclusions may be made about the relationships between values of the

$$\frac{Soil {Al}_{2} {O}_{3}}{Soil {SiO}_{2}}$$

ratio and the soil texture of a soil sample.

For example:
If the

$$\frac{Soil {Al}_{2} {O}_{3}}{Soil {SiO}_{2}}$$

ratio equals 0.04, then the soil texture is loamy sand.
If the ratio equals 0.08, then the soil texture is sandy loam.
If the ratio equals 0.18, then the soil texture can be clay loam, loam, or silty loam.
If the ratio equals 0.3, then the soil texture is clay or silty clay, etc.

Although the value of the

$$\frac{Soil {Al}_{2} {O}_{3}}{Soil {SiO}_{2}}$$

ratio may not provide definitive identification of the soil texture type in all cases, the soil texture class (ie: whether the soil is predominantly sandy, loam, clay, etc.) may be determined. Therefore, in one aspect of the present disclosure, a relatively fast, non-destructive, in-situ method for determining soil elemental content and the

$$\frac{Soil {Al}_{2} {O}_{3}}{Soil {SiO}_{2}}$$

ratio is provided, and either the soil texture type or the soil texture class may be determined.

### Gamma Spectra and the Tagged Neutron Method of Neutron-Gamma Analysis

Neutron-gamma analysis is based on the measurement of gamma ray response that appears during fast neutron irradiation of a studied object, such as soil. After colliding with a neutron (either fast neutrons, or moderated to thermal energy), the nuclei of soil elements undergo specific reactions and emit gamma rays of a specified energy. The intensity of these gamma rays is proportional to the concentration of the element undergoing the reaction in analyzed soil. By comparing the registered gamma spectrum with reference data, soil composition can be determined.

When a material is hit with a neutron ray, it produces Inelastic Neutron Scattering (INS) gamma rays and Thermal Neutron Capture (TNC) gamma rays. When it comes to measuring the content of different oxides in the soil, for example the oxides containing Si or Fe, it is the INS gamma rays that produce distinctive peaks for identifying these elements. Therefore, to accurately measure the ratios of different oxides present in a soil, it is required to obtain INS gamma spectra that are relatively clean, with a low signal-to-noise ratio.

One method for obtaining INS spectra of a soil sample is to utilize Pulsed Fast Thermal Neutron Analysis (PFTNA). By pulsing PFTNA, when the neutron generator is pulsed “on”, all three types of gamma rays are produced, including INS, TNC and Delayed Activation (DA). When the neutron generator is pulsed “off”, only TNC and DA gamma rays are produced. Therefore, a clean INS signal may be obtained by subtracting the measured gamma ray spectrum when the neutron generator is on, from the measured gamma ray spectrum when the neutron generator is off. From the INS spectra of the soil sample, the content of targeted elements, such as Fe and Al, that are present in the soil, may be determined. The oxide ratios of the soil may then be calculated, as described herein.

A mobile PFTNA system for acquiring gamma spectra and measuring moisture content includes a pulsed neutron generator, a scintillation detector for detecting the gamma rays reflected from the soil, a power source and electronics for operating the system. A pulsed neutron generator is used as a neutron source; an example of a pulsed neutron generator that may be used for this purpose is the model MP 320 portable neutron generator manufactured by Thermo Fisher Scientific. The gamma rays are registered by scintillation detectors; in an example embodiment of the apparatus, three large-volume sodium iodide (NaI) crystal scintillator detectors (having a total volume of approximately 7.5 L) may be used, such as NaI gamma detectors manufactured by Scionix. One example of a detector assembly comprises an NaI crystal coupled to a photomultiplier tube (PMT). The NaI detector assemblies may be provided with corresponding electronics, such as manufactured by XIA LLC. A power system for powering the apparatus may comprise, for example, four 12V batteries, an DC-AC inverter, and a charger. Optionally, the apparatus may be provided with a GPS device, which provides geographical coordinates of the system during scanning operations. The system may be operated by a computing device, such as a laptop or other suitable computing device as would be known to a person skilled in the art. Optionally, the system may be mounted to a mobile cart and pulled by a vehicle, such as a tractor, for obtaining a plurality of gamma spectra of soil across a large area, such as a field.

In another embodiment, the Tagged Neutron Method (TNM), also referred to as Associated Particle Imaging, is a technique for neutron-gamma analysis with an improved signal-to-background ratio. In TNM, a neutron generator produces 14.1 MeV neutrons through the t(d,n)α reaction. The created neutrons are accompanied by alpha particles, which serve as “tags” for the neutrons. The gamma spectra are recorded in alpha-gamma coincidence mode and represent the spectrum exclusively of the soil sample (or other sample being measured). From such TNM spectra, determining the chemical composition and content of the irradiated object (such as soil) is relatively straightforward. In TNM, INS gamma rays created due to fast neutron interaction with the nuclei of soil elements are registered. The type of reaction, cross-section, and energy of gamma rays for main soil elements are presented in Table 2, below. The INS gamma peaks with energy listed in Table 2 may be found in the soil gamma spectra. Other peaks (from listed, or other, nuclei present in the soil) will be of very low intensity, and their registration is unlikely. Comparing the registered gamma spectrum with reference spectra of separate components allows for the determination of elemental composition in the soil.

TABLE 2
Types of fast neutron-nuclei reactions, cross-sections,
and energies of gamma rays for main isotopes (and
their natural abundance) of primary soil elements

| | | | Cross- | | |
|---|---|---|---|---|---|
| | | | section, | | |
| | | | mb | Gamma | |
| | Abun- | | (neutron | ray | |
| Iso- | dance | | energy | energy, | Refer- |
| tope | % | Reaction | 14 MeV) | MeV | ence |
| ¹²C | 98.9 | ¹²C(n, | 210 | 4.439 | NNDC, |
| | | n′)¹²C*→¹²C + γ | | | 2020 |
| ¹⁶O | 99.8 | ¹⁶O(n, | 148 | 6.129 | Simakov |
| | | n′)¹⁶O*→¹⁶O + γ | 38 | 2.742 | et al., |
| | | | 47 | 6.917 | 1998 |
| | | | 53 | 7.117 | |
| | | ¹⁶O(n, | 17.2 | 4.439 | |
| | | n′α)¹²C*→¹²C + γ | | | |
| | | ¹⁶O(n, | 57 | 3.684 | |
| | | α)¹³C*→¹³C + γ | 34 | 3.854 | |
| | | | 22 | 3.089 | |
| ²⁸Si | 92.3 | ²⁸Si(n, | 120 | 1.778 | NNDC, |
| | | n′)²⁸Si*→²⁸Si + γ | | | 2020 |
| ²⁷Al | 99.9 | ²⁷Al(n, | 9 (2 | 0.844 | NNDC, |
| | | n′)²⁷Al*→²⁷Al + γ | MeV - | | 2020 |
| | | | 100) | | |
| | | | 17 (2 | 1.014 | |
| | | | MeV - | | |
| | | | 230) | | |
| | | | 32 (3 | 2.211 | |
| | | | MeV - | | |
| | | | 240) | | |
| | | | 23.5 (4.5 | 3.004 | |
| | | | MeV- | | |
| | | | 180) | | |
| | | ²⁷Al(n, | 184 | 1.810 | Simakov |
| | | d)²⁶Mg*→²⁶Mg + γ | | | et al., |
| | | | | | 1998 |
| ⁵⁶Fe | 91.7 | ⁵⁶Fe(n, | 621 | 0.847 | Simakov |
| | | n′)⁵⁶Fe*→⁵⁶Fe + γ | 290 | 1.238 | et al., |
| | | | | | 1998 |
| ⁴⁰Ca | 96.9 | ⁴⁰Ca(n, | 35 | 3.737 | NNDC, |
| | | n′)⁴⁰Ca*→⁴⁰Ca + γ | 5 | 3.904 | 2020 |
| | | ⁴⁰Ca(n, | 152 | 1.611 | |
| | | α)³⁷Ar*→³⁷Ar + γ | | | |
| ²⁴Mg | 79 | ²⁴Mg(n, n′)²⁴Mg*→ | 364 | 1.369 | Simakov |
| | | ²⁴Mg + γ | 100 | 1.809 | et al., |
| | | | | | 1998 |

Legend: n—neutron, α—alpha particle, d—deuteron, γ—gamma ray, and *—exited nuclei.

### TNM Mobile System Design, Arrangement, and Data Acquisition

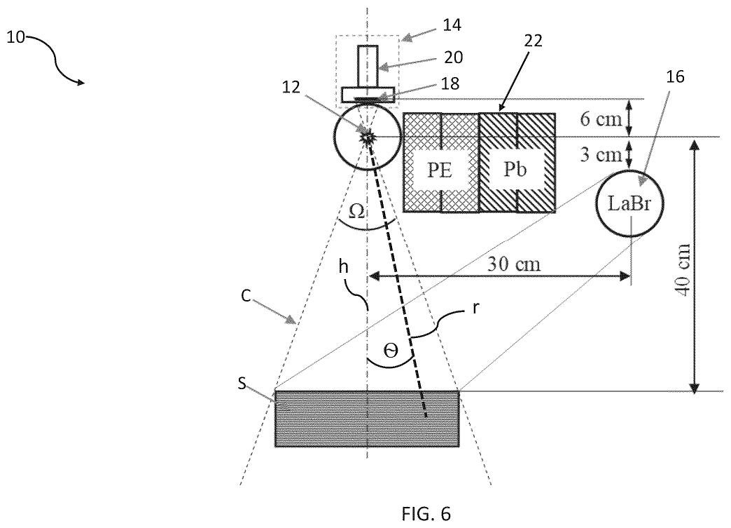

Referring to FIG. 6, an example embodiment of a TNM system 10 for measuring gamma spectra in TNM includes the following main components:
Neutron generator 12 with an alpha scintillator 18, photomultiplier tube (PMT) 20 and an alpha detector 14;
Gamma detector 16, the gamma detector 16 spaced apart from, and positioned laterally of, the neutron generator 12;
Operational electronics and laptop (not shown);
Radiation shielding 22, such as may be constructed from polyethylene (PE) and lead (Pb), to screen the gamma detector 16 from direct neutron irradiation.

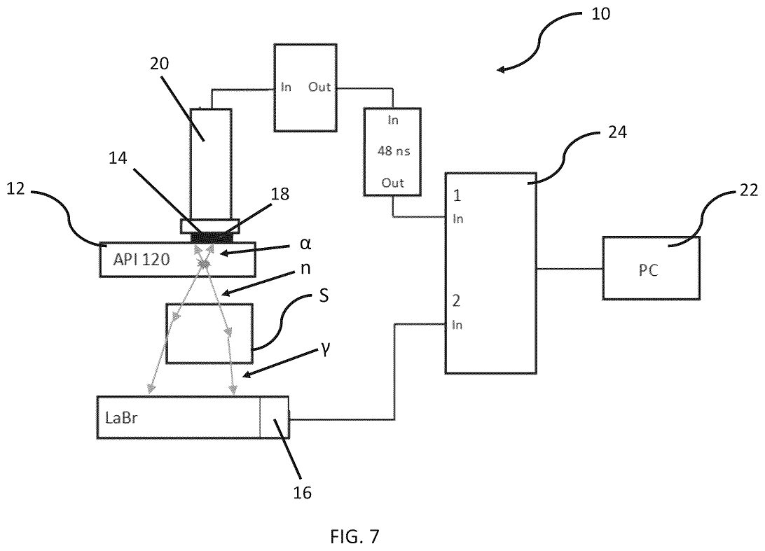

Additional details of components of an example TNM system 10 are shown in FIG. 7. These specific components are provided as illustrative examples only, and are not intended to be limiting:
Neutron generator 12 may be an API120 portable neutron generator with built-in alpha detector 14 (Thermo Fisher Scientific, CO), which provided up to 2·10⁷ n·s⁻¹ neutron flux (max accelerator voltage 90 kV, max beam current 50 ρA) with an energy of 14.1 MeV;
An alpha detector consisting of a YAP scintillator 18 (Yttrium Aluminum Perovskite crystal doped by Cerium, YAP (Ce), crystals) with a Hamamatsu R13089 fast photomultiplier (PMT) 20;
A cerium-doped Lanthanum Bromide crystal (LaBr3(Ce)) gamma detector 16 (scintillation LaBr3(Ce) crystal sizes having a diameter of 89 cm and a height of 203 cm, Saint-Gobain, France) for gamma ray measurement;
A 4-channel digital pulse processor 22, for example in a desktop computer or other suitable computing device, with integrated Linux operating system, Pixie-Net (Pixie-Net, 2024) as operational electronics 24 for detecting radiation; and
Polyethylene-lead shielding (not shown in FIG. 7), installed between the neutron generator 12 and gamma detector 16.

The TNM system 10 may be built on a mobile platform, such as on a tractor and trailer. The TNM system 10 may also be used for both laboratory measurement of large samples and for field measurements, wherein the TNM system is moved to spots on the field where point measurements are taken in a point sampling mode at discrete locations spread across the field, rather than continually taking measurements across the field in a scanning mode, such as is accomplished with the PFTNA system. If it is only required to measure soil samples in a lab, optionally, the TNM system does not need to be mobile, and the components may simply be mounted to a frame or housing. For applications requiring a mobile TNM system, in addition to mounting the TNM system components to a mobile platform (such as a trailer that will be pulled by a tractor), the TNM system may additionally include a Global Positioning System (GPS) for correlating each discrete gamma spectrum of the plurality of gamma spectra, obtained by the mobile TNM system, to a geographic location. The inclusion of a GPS in the TNM system allows for the measurement and identification of variations in soil texture that may occur at point measurements taken across a field or other large area of soil to be classified.

Radiation shielding may be constructed of any suitable material for shielding the gamma detector from neutron radiation emitted directly from the neutron generator. If the gamma detector 16 receives neutrons emitted directly from the neutron generator, rather than gamma rays emitted from the irradiated soil sample, such stray neutron radiation would interfere with the ability of the gamma detector to measure gamma rays and would introduce noise into the signal. As an example, without intending to be limiting, the radiation shielding may be constructed of borated polyethylene (ie: boron incorporated into high-density polyethylene (HDPE)); borated-lead polyethylene (ie: boron and lead incorporated into HDPE); and/or lead. Radiation shielding constructed of HDPE is an option that offers decreased weight while providing the required level of radiation shielding, and as such, may be particularly suited for mobile gamma detection systems.

With reference to FIGS. 6 and 7, the neutron generator 12 generates neutrons (n) and alpha particles (α) in approximately opposite directions; for example, the angle between the alpha particles (α) and the neutron rays (n) may be nearly 180°, and in some laboratory coordinate system studies, this angle has been determined to be approximately 174°. When the neutrons (n) hit the sample (S), gamma rays (γ) are produced. The alpha particle (α) is detected by an alpha detector 14 (or in other words, the alpha particle gauge pulse moment) within a narrow coincidence cone C, the cone C having an apex originating from the neutron source 12 and directed upward to form a solid angle Ω of approximately 0.44 steradian. The pulse from the alpha detector 14 initiates the Pixie-Net module (run type 0x400), which then waits for a short period for the arrival of the gamma pulse from the gamma detector 16. For example, not intended to be limiting, the example TNM system described herein is configured so that time period to wait for the arrival of the gamma pulse is set to 40 ns. During this time, the neutron paired with the detected alpha particle travels to the sample within a cone C with an apex on the neutron target and directed downward (14.1 MeV neutron speed is 5.2 cm ns⁻¹, leading to a travel time of approximately 8-10 ns to travel the distance to the sample S). The neutron (n) hits the sample (S) creating a gamma ray (γ) that is registered by the gamma detector 16, with the resulting gamma spectrum saved by the Pixie-Net module 24. Optionally, this process may be repeated as the system 10 is moved across a field in a point sampling mode.

The Pixie-Net module 24 creates the event records in binary format; for example, the file size may be in the range of 1 to 2 GB, depending on the measurement time. A Time-Of-Flight (TOF) spectrum is generated, which shows the dependence of gamma ray counts (ie: number of gamma rays registered by the detector 16) over a period of time, with time t=0 being the alpha particle gauge pulse moment.

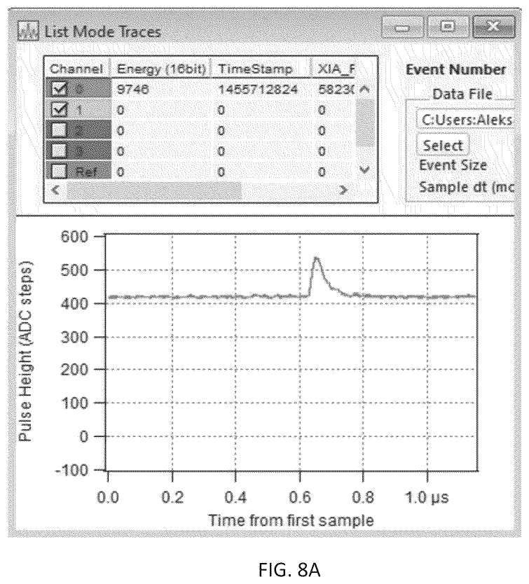

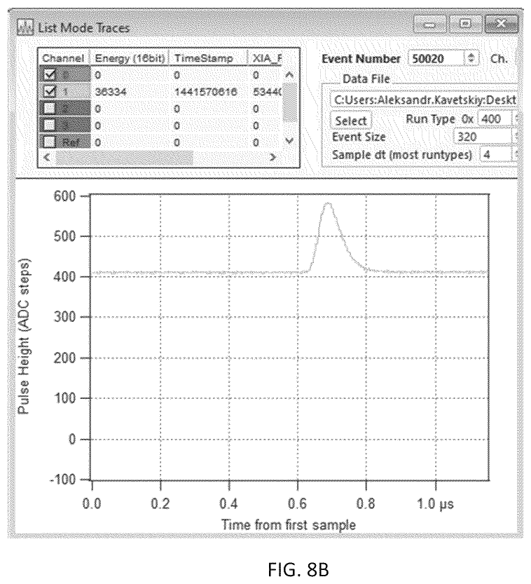

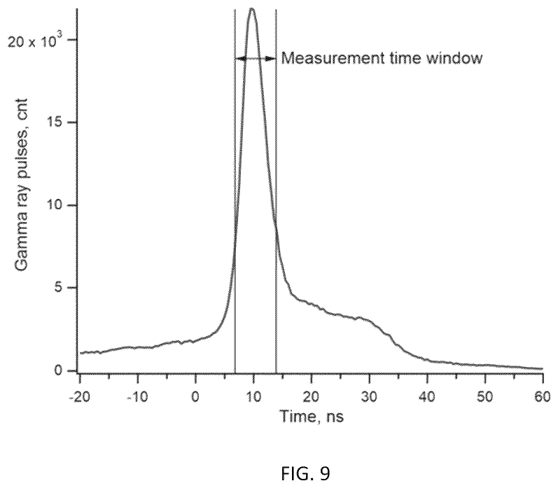

The gamma spectrum may be generated from this file using suitable software applications; for example, not intended to be limiting, the IGOR™ software application (WaveMetrics, 2017 with XIA™ firmware updates implemented) may be used to generate the gamma spectrum from the TOF spectrum. Screenshots from the IGOR™ software, providing examples of saved alpha and gamma pulses, are shown in FIGS. 8A and 8B. FIG. 9 shows an example of a TOF spectrum generated by the IGOR™ software. These gamma spectra (acquired within a narrow time window around the location of the peaks in the TOF spectrum) can be attributed exclusively to INS processes occurring in the sample after exposure to the neutron particles. Thus, these gamma spectra may be used for determining sample mass fractions, correlating to the content of different elements found in the soil sample through the deconvolution process, as will be further explained below.

### Soil Gamma Spectrum Deconvolution on Spectra of Soil Components

As mentioned herein, soil is a mixture of oxides (SiO₂, Al₂O₃, Fe₂O₃, CaO, MgO) along with carbon and water. The soil gamma spectrum may be conceptualized as the summation of the gamma spectra produced by each of the individual components within a soil sample. The deconvolution process is a mathematical procedure that allows for identifying the contribution of each soil component to the total gamma spectra produced by a soil sample. The relative contribution to the gamma spectra by each soil component is proportional to the mass fraction of each soil component. The deconvolution process, described herein, may be performed on INS spectra obtained of a soil sample, regardless of whether the INS spectra is obtained by a TNM system or a PFTNA system. In the discussion of the experimental results and analysis discussed herein, it will be appreciated that although the experimental results were obtained by a TNM system, that the methods and equations described below may also be performed on INS gamma spectra obtained using a PFTNA system, and that INS gamma spectra of a soil obtained by a TNM system, a PFTNA system, or any other system, is intended to be included in the scope of the present disclosure.

As previously mentioned, the soil gamma spectrum can be approximated by summing component gamma spectra while accounting for their mass fractions. To reach the quantified agreement between the soil spectrum and sum spectrum of components it needs to take into account that neutron stimulated gamma spectra of soil and reference samples depend on the density of samples, their volume and attenuation of radiation (neutron and gamma) into the body of samples. So, the volume of the soil and reference samples should be approximately the same, the spectra should be converted to the non-attenuation condition of measurement and compared spectra should be normalized to the density of samples equal 1 g/cm³. In this case the next equation may be written as:

$$\frac{{R}_{ss no att , i}}{{d}_{ss}} \approx {\sum }_{j} {w}_{j} \cdot \frac{{G}_{j no att , i}}{{d}_{j}} \qquad ( 1 )$$

where:
R_{ss no att,i} is the spectrum of a soil with density dₛₛ restored to non-attenuation conditions;
G_{j,no att,i} is spectra of the j soil component with density dⱼ restored to non-attenuation conditions;
wⱼ is the mass fraction of the jₜₕ component in soil; and
i is channel number in spectra.

In an aspect of the present disclosure, two conditions should be met to apply the deconvolution procedure for component mass fraction determinations:
1. All measurements should be done under the same geometrical conditions, meaning that all the measurements should be performed by the same system 10, with the same relative distance and angular positioning of the neutron generator 12, gamma detector 16 and soil sample S, and furthermore, the measurements should be performed on samples having the same volume; and
2. The measured spectra of soil and reference samples should be corrected for radiation attenuation into the sample body. Due to self-adsorption of radiation into the body of the relatively large soil samples being measured, spectra measured from the same materials having different densities, will yield differences in the measured gamma spectra obtained from such samples. Therefore, in consideration of this effect of self-adsorption of the radiation, methods described herein are used to restore the spectra to a “non-attenuated condition” using equations 2 and 3.

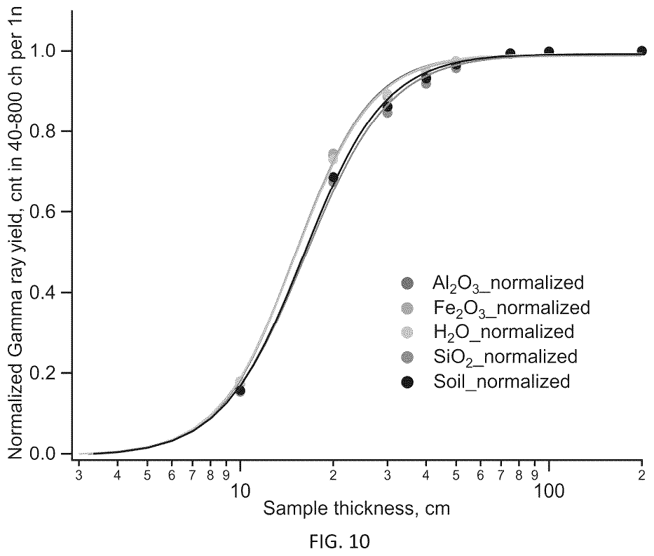

When measuring gamma spectra of soil in the field, the soil volume may be approximated to be semi-infinite. Each of the soil components at measurement should, ideally, be present in equal volumes within the soil sample; however, this is not the case in naturally occurring soil samples. Therefore, the dependence of gamma spectra intensity of each oxide component in a soil, versus the sample volume of that oxide component in the soil, was examined by Monte-Carlo gamma spectra computer simulation of different soil samples, each soil sample containing different oxides with varying volumes. For example, the computer software program MCNP6.2 (MCNP6.2, 2017) was used for the computer simulations. The design of the modeled measurement system was similar to the experimental TNM system 10 described above. Sample volume was represented by a cylinder having a radii t and a thickness t. Simulations showed that spectra intensity initially increased with increasing t and reached steady state level at t>50 cm (volume of sample ˜0.4 m³). The resulting simulated dependencies of simulated spectra intensity versus thickness of the cylindrical sample are shown in FIG. 10 for different oxides. Thus, measuring the gamma spectra of oxides which will be subsequently analyzed by performing the deconvolution process, should be done using sample volumes of no less than 0.4 m³ (for example, the sample may be contained in a 1 m×1 m×0.5 m box). Subsequent measurements of soil component gamma spectra, described herein, were done at the same sample volume. Similarity of all geometrical conditions (such as, the distance between the various TNM or PFTNA system components and the sample, the sample volume, and the angles of the neutron beam relative to the norm), were attained.

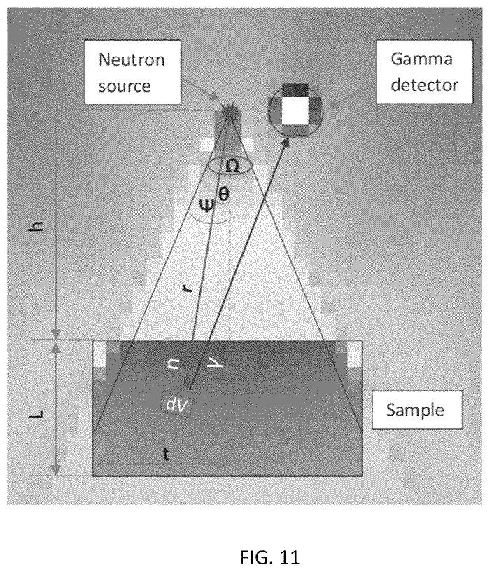

To account for radiation attenuation, all measured spectra were converted to the non-attenuation condition. This was done using the following equations according to Kavetskiy, A., Yakubova, G., Prior, S. A., Torbert, H. A., 2024, “Carbon analysis of large soil samples using the tagged neutron method: Accounting for radiation attenuation” Appl. Radiat. Isotopes 209:111332. https://doi.org/10.1016/j.apradiso.2024.111332 (hereinafter, “Kavetskiy, 2024”):

$${R}_{ss no att , i} = {R}_{ss , i} \cdot \frac{\int \frac{1}{{r}^{4}} dV}{\int \frac{1}{{r}^{4}} \cdot exp [ - ( {\sum }_{total , SS} + {μ}_{lin , SS} ( {E}_{i} ) ) \cdot ( r - \frac{h}{cos θ} ) ] dV} \qquad ( 2 )$$

$${G}_{j no att , i} = {G}_{j , i} \cdot \frac{\int \frac{1}{{r}^{4}} dV}{\int \frac{1}{{r}^{4}} \cdot exp [ - ( {\sum }_{total , j} + {μ}_{lin , j} ( {E}_{i} ) ) \cdot ( r - \frac{h}{cos θ} ) ] dV} \qquad ( 3 )$$

where:
R_{ss,i} is the measured spectrum of a soil;
G_{j,i} is spectra of the j soil component (reference sample);
All integrals are taken by volume of sample;
r, h, θ, dV are geometrical parameters shown on the calculation scheme (as shown in FIG. 11);
Σ_{total,j} and Σ_{total,ss} are total macroscopic cross-section of 14.1 MeV neutron interactions with nuclei of components (j) and nuclei of soil (SS); and
μ_{lin,ss}(Eᵢ) and μ_{lin,j}(Eᵢ) are linear coefficient attenuation gamma rays with energy Eᵢ in body of components (j) and soil (SS).

The component materials used for deconvolution of soil spectrum were oxides, carbon and water. Thus, the macroscopic neutron cross-section of component material can be found as:

$${\sum }_{total , j} = ( \frac{{σ}_{total , j} \cdot {N}_{Av}}{{Aw}_{j}} \cdot {t}_{j} + \frac{{σ}_{total , O} \cdot {N}_{Av}}{16} \cdot {t}_{O , j} ) \cdot {d}_{j} \qquad ( 4 )$$

where:
σ_{total,j} is a total of 14.1 MeV neutron cross-section with metal or hydrogen in oxides and for carbon;
Awⱼ is atomic weight;
tⱼ, t_{O,j} are the mass fractions of the j metal or hydrogen in oxides and oxygen, respectively; for Carbon, these values are tⱼ=1, t_{O,j}=0;
σ_{total,O} is a total of 14.1 MeV neutron cross-section with oxygen;
N_{Av} is the Avogadro number.

Values of σ_{total,j} and σ_{total,O} can be found in an available database (NNDC. 2020).

Linear coefficient attenuation gamma rays with energy E; in the body of components j may be found as:

$${μ}_{lin , j} ( {E}_{i} ) = [ {μ}_{j} ( {E}_{i} ) \cdot {t}_{j} + {μ}_{O} ( {E}_{i} ) \cdot {t}_{O , j} ] \cdot {d}_{j} \qquad ( 5 )$$

where:
μⱼ(Eᵢ) is mass attenuation coefficient of metal or hydrogen in oxide and carbon with energy;
μ_{O}(Eᵢ) is oxygen mass attenuation coefficient with energy.

Mass attenuation coefficients may be found in the NIST database (NIST, 2018).

Taking into account that soil is represented as the sum of oxides, water and carbon, Σ_{total,ss} may be calculated as:

$${\sum }_{total , SS} = {d}_{SS} \cdot {\sum }_{j} {w}_{j} \cdot \frac{{\sum }_{total , j}}{{d}_{j}} \qquad ( 6 )$$

and μ_{lin,ss}(Eᵢ) may be calculated as:

$${μ}_{lin , SS} ( {E}_{i} ) = {d}_{SS} \cdot {\sum }_{j} {w}_{j} \cdot \frac{{μ}_{lin , j} ( {E}_{i} )}{{d}_{j}} \qquad ( 7 )$$

The least squares method may be applied for determining component mass fractions, as shown in Equation (8) below. Considering the above, an equation for finding wⱼ may be written as follows:

$${\sum }_{i} {( \frac{{R}_{ss no att , i}}{{d}_{ss}} - {\sum }_{j} {w}_{j} \cdot \frac{{G}_{j no att , i}}{{d}_{j}} )}^{2} \to min \qquad ( 8 )$$

Some computer algorithms and standard software may be used to solve Equation (8). For example, without intending to be limiting, the Levenberg-Marquart algorithm implemented in Mathematica (Mathematica, 2023) may be used.

Thus, having the gamma spectra of soil and the component oxides that make up the soil, and applying the deconvolution procedure while accounting for radiation attenuation, the soil content in terms of mass fractions of each soil component (oxides) may be determined.

To examine the feasibility of the developed methodology, several soils modeled as mixtures of oxides were virtually created and gamma spectra of oxides under neutron irradiation were simulated using the Monte-Carlo computer method (Hendricks, J. S., 1994, “A Monte Carlo code for particle transport” Los Alamos Science 22, 31-43 (hereinafter, “Hendricks, 1994”)). The widely used computer software package MCNP6.2 (MCNP User's Manual, code version 6.2, https://mcnp.lanl.gov/pdf_files/TechReport_2017_LANL LA-UR-17-29981_WernerArmstrongEtAl.pdf (accessed 14 Nov. 2024)) was applied for this purpose. Then the deconvolution procedure was conducted to determine the modeled soil content. Results were compared with data (oxides content) used in the soil modeling. These comparisons are presented below in Table 3.

As can be seen, for thin samples (for example, having a thickness of 1 cm), accounting for radiation attenuation in the deconvolution procedure is not required. However, for thick samples, accounting for radiation attenuation in the deconvolution procedure yields much better agreement with content values used when creating soil models, as compared to using the same procedures without accounting for radiation attenuation. Accordingly, in one aspect of the present disclosure, the experimentally measured TNM or PFTNA gamma spectra (obtained from real-world soil samples or fields) may be processed using deconvolution procedures that account for radiation attenuation when determining soil content.

TABLE 3
Composition of modeled soil and content of components received from the deconvolution
procedure using Monte-Carlo computer-simulated gamma spectra

| | | | | Results of soil content determination | | | |
|---|---|---|---|---|---|---|---|
| Model characteristic | | | | Accounting for | | Not accounting for | |
| Soil | Layer | | | radiation | | radiation | |
| density | thickness | | wⱼ, | attenuation | | attenuation | |
| (g/cm³) | (cm) | Component | wt % | wⱼ, wt % | Δwⱼ, wt % | wⱼ, wt % | Δwⱼ, wt % |
| 1.3 | 1 | SiO₂ | 50 | 49.6 | −0.4 | 50.4 | +0.4 |
| | | Al₂O₃ | 40 | 39.7 | −0.3 | 39.4 | −0.6 |
| | | C | 10 | 9.9 | −0.1 | 10 | 0 |
| 1.3 | 50 | SiO₂ | 72 | 71.5 | +0.5 | 79.0 | −7.0 |
| | | Al₂O₃ | 18 | 19.8 | −1.8 | 14.0 | +4.0 |
| | | C | 10 | 9.0 | +1.0 | 7.1 | +2.9 |
| 1.3 | 100 | SiO₂ | 48 | 46.9 | +1.1 | 41.5 | +6.5 |
| | | Al₂O₃ | 24 | 24.1 | −0.1 | 23.2 | +0.8 |
| | | C | 8 | 7.1 | +0.9 | 9.6 | −1.6 |
| | | H₂O | 20 | 22.0 | −2 | 25.7 | −5.7 |

### Soil Bin and Field Soil Characteristics-Experimental Results

To test the efficiency of the developed methodology for determining soil chemical composition and soil texture, TNM measurements of soil bins at the USDA-ARS National Soils Dynamics Laboratory (NSDL) and on some real agricultural fields with known soil textures were conducted. These bins were ˜80 m long, ˜6 m wide, and ˜0.6 m deep and were filled with representative soils found in the southeastern US. The sand, clay, and silt content, soil texture, and mineral content of these bins were previously characterized (Batchelor, 1984) and are shown in Table 4 below. The analysis of soil component content (sand, clay, silt) and soil texture for each of the real agricultural fields that were measured (Pitt Place, Curt Cope), was completed using laboratory analysis of soil samples from each field; the results of this laboratory analysis is also included in Table 4.

TABLE 4
The content of sand, clay and silt and soil texture
on the bins at the USDA-ARS NSDL (Batchelor, 1984)

| Bin# or Field | Soil component content, wt % | | | |
|---|---|---|---|---|
| Name | Sand | Clay | Silt | Soil texture |
| Indoor bin | 71.6 | 11 | 17.4 | Sandy loam |
| Bin-3a | 5.1 | 62.5 | 32.4 | Clay |
| Bin-3b | 20.6 | 61.1 | 18.3 | Clay |
| Bin-4 | 73.1 | 16 | 10.9 | Sandy loam |
| Bin-5 | 24.9 | 44.2 | 30.9 | Clay |
| Bin-7 | 9.3 | 46 | 44.7 | Silty clay |
| Bin-8 | 23.2 | 59.6 | 17.2 | Clay |
| Bin-9a | 5.5 | 66.4 | 28.1 | Clay |
| Bin-9b | 1.6 | 57.2 | 41.2 | Silty clay |
| Lab Field | 80 | 5 | 15 | Loamy sand |
| Pitt Place1 | 78 | 3 | 19 | Loamy sand |
| Pitt Place2 | 38 | 27 | 35 | Clay loam |
| Pitt Place3 | 87 | 5 | 8 | Sand |
| Curt Cope4 | 50 | 25 | 25 | Sandy clay loam |
| Curt Cope5 | 43 | 33 | 24 | Clay loam |
| Curt Cope6 | 75 | 6 | 19 | Sandy loam |

Batchelor, J. A., Jr. (1984); Properties of Bin Soils at the National Tillage Machine Laboratory, Pub. 218. Auburn, AL: USDA-ARS National Soil Dynamics Laboratory (herein, “Batchelor, 1984”).

### Comparison of the TNM Method and Other Methods

Results of soil elemental content measurements obtained using the TNM methods described herein, wherein the INS gamma spectra of the soil were obtained using a TNM system, were compared with those obtained using other techniques, including chemical analysis for carbon and silicon content, time domain reflectometry, and a nuclear method for moisture. Throughout this disclosure, although the experimental results described herein involved obtaining INS gamma spectra using a TNM system, it will be appreciated that INS gamma spectra obtained by any other means, such as by using a PFTNA system, may also be used in the systems and methods disclosed herein to identify the soil texture classification of a soil. References below, to the “TNM system” and the “TNM method”, in describing the experimental results obtained using a TNM system, are intended to apply equally to using INS gamma spectra of the soils by any other system or method, and it will be appreciated that the methods and systems disclosed herein are not intended to be limited to utilizing INS gamma spectra obtained by a TNM system. Bland-Altman (Bland et al., 1999) and Deming regression (SPC, 2024) plots were generated for this comparison.

The Bland-Altman (Giavarina, 2015) plot displays the differences between values obtained from two comparable methods versus the average of those values. Agreement between the two methods can be concluded based on the following criteria (Giavarina, 2015):
The mean of the differences is close to zero,
95% of the differences fall within the range ‘Mean±1.96×STD’,
The distribution of the differences is approximately normal.

To assess normality, the Jarque-Bera (JB) test can be applied. The null hypothesis of normal distribution cannot be rejected if the JB statistic is less than the critical value.

Deming regression is a statistical technique used to fit a line to two-dimensional data where both variables are subject to measurement errors. It is commonly employed in method comparison studies to assess the agreement between different measurement techniques. Several standard software packages support Deming regression analysis; in this study, SPC for Excel™ (SPC, 2024) was used. This software generated the Deming regression line for the two data sets, calculating the regression coefficients (slope and intercept), their standard errors, t-statistics, p-values (indicating the probability that the t-statistic would be observed under the null hypothesis), and the lower and upper confidence limits (LCL and UCL) at a significance level of α=0.05.

Two hypothesis tests were performed in this statistical analysis:
1. Slope Test:
   Null hypotheses H₀: Slope−1=0;
   Alternative hypothesis H₁: Slope−1≠0.

If the slope test yields a high p-value (>0.05) and the 95% confidence interval includes zero, the null hypothesis (H₀) cannot be rejected, indicating no significant difference from a slope of 1.
2. Means Test:
   H₀: The difference in the means of the two methods is 0;
   H₁: The difference in the means is not 0.

Similarly, if the p-value is high and the confidence interval includes zero, H₀ cannot be rejected, suggesting that the two methods yield equivalent mean values.

When both the slope and means tests fail to reject the null hypotheses, it supports the conclusion that the two measurement methods are comparable. In this study, both the Bland-Altman plot and the Deming regression analysis were used to compare TNM results with those obtained by other methods.

### Soil Bin and Field Soil Oxide Contents Defined by TNM Measurements

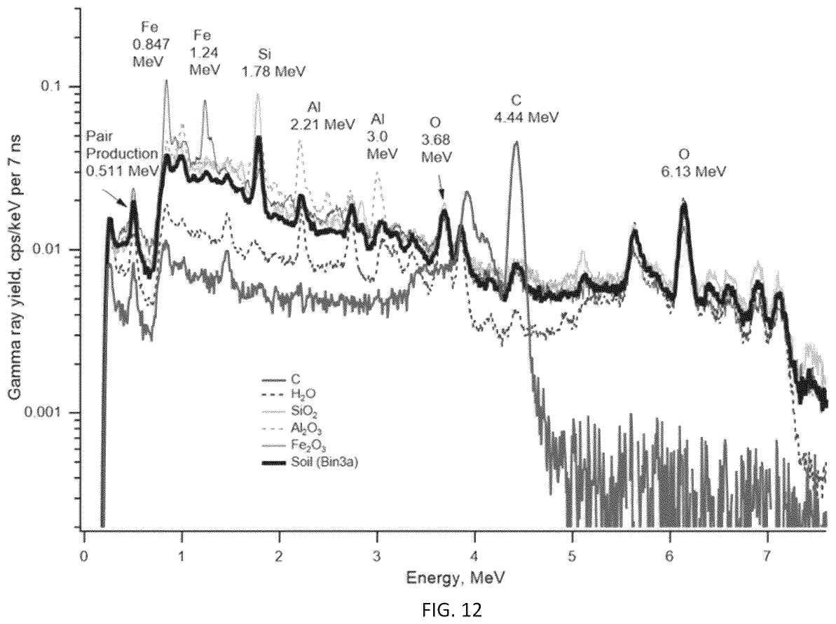

To provide an illustrative example, the gamma spectra of reference oxides measured by the TNM system, using samples with volumes of around 0.5 m³, are shown in FIG. 12, along with the gamma spectrum measured by the TNM system on soil bin 3a, characterized in Table 4 above (the gamma spectrum obtained from measurement of the soil bin 3a represented in FIG. 12 by the bold black curve). As may be seen from the gamma spectra provided in FIG. 12, the gamma peaks present in the gamma spectrum of the soil sample in bin 3a may be observed in one or another reference oxide spectrum. This indicates that all elements present in the soil bin 3a are attributable to one of the reference oxides provided in FIG. 12.

The deconvolution procedure was applied to the soil bin gamma spectra measured by the TNM system 10. For the deconvolution procedure using Equation 9, densities of studied objects are required. Densities of references oxides were measured by the weight method, while soil bin densities were measured by the nuclear method using a Model 3440 Moisture Density Gauge (Troxler Inc., Research Triangle Park, NC).

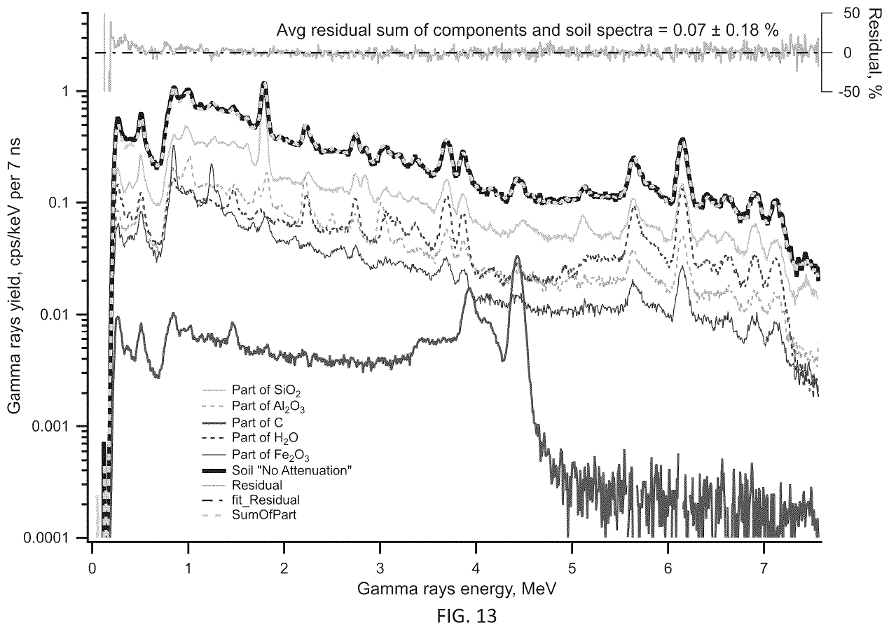

An example of deconvoluting the TNM gamma spectrum for one soil bin, using gamma spectra of its components (taken over a 7 ns time window), and the sum of components spectrum are shown in FIG. 13. This figure shows the spectra of the individual oxide reference samples according to their mass fractions in soil, a summed spectrum of these components, measured spectrum of the soil Bin 3a, and the residual between the summed spectrum and the measured soil spectrum. As can be seen, the summed spectrum fully coincided with spectrum of the measured soil Bin 3a, and the average residual value was very close to zero. The results of performing the deconvolution procedure on real-world gamma spectra taken of a soil bin sample supports the correctness of the soil spectrum deconvolution procedure.

The mass fraction of reference oxides, carbon, and moisture for all surveyed soil bins are shown in Table 5. Note that the mass fractions of all components were received as results of applying the deconvolution procedure. Moisture content (mo) in Table 5 was calculated as:

$$mo = \frac{{w}_{{H}_{2} O}}{1 - \frac{{w}_{{H}_{2} O}}{100}} \qquad ( 9 )$$

while other component contents were calculated relative to dry soil as:

$${w}_{dry , j} = \frac{{w}_{j}}{1 - \frac{{w}_{{H}_{2} O}}{100}} \qquad ( 10 )$$

These data (dry soil basis) may be used independently from soil moisture. The elemental (Si, Al, C, Fe) content in soil, Elⱼ, may be calculated as:

$${El}_{j} = {w}_{dry , j} \cdot {t}_{j} \qquad ( 11 )$$

and oxygen content in dry soil (excluding oxygen in water), ( ) may be calculated as:

$$O = {\sum }_{j} {w}_{dry , j} \cdot {t}_{O , j} \qquad ( 12 )$$

TABLE 5
Soil component contents in soil bins and in fields determined by using the deconvolution
$procedure for TNM gamma spectra and the \frac{Soil {Al}_{2} {O}_{3}}{Soil {SiO}_{2}} ratio calculated from presented data$

| | Moisture, | Component contents in dry soil, wt. % | | | | |
|---|---|---|---|---|---|---|
| Bin# or soil spot name | % (±2.0 %) | SiO₂ (±6.0 wt. %) | Al₂O₃ (±2.3 wt. %) | C (±0.7 wt. %) | Fe₂O₃ (±4.7 wt. %) | $\frac{Soil {Al}_{2} {O}_{3}}{Soil {SiO}_{2}}$ |
| Indoor bin | 0 | 85.6 | 9.3 | 0 | 5.1 | 0.109 |
| Bin-3a | 26.6 | 56.0 | 25.7 | 3.6 | 14.8 | 0.459 |
| Bin-3b | 24.5 | 59.9 | 15.7 | 4.2 | 20.2 | 0.262 |
| Bin-4 | 6.9 | 87.4 | 7.7 | 1.4 | 3.4 | 0.088 |
| Bin-5 | 15.5 | 61.2 | 22.1 | 1.6 | 15.2 | 0.361 |
| Bin-7 | 23.4 | 65.8 | 18.2 | 2.9 | 6.5 | 0.277 |
| Bin-8 | 15.8 | 58.1 | 27.2 | 2.1 | 12.6 | 0.468 |
| Bin-9a | 30.3 | 60.2 | 19.6 | 4.2 | 15.9 | 0.326 |
| Bin-9b | 25.2 | 59.5 | 21.7 | 3.6 | 15.2 | 0.365 |
| Lab field | 10.3 | 73.9 | 16.1 | 2.5 | 8.0 | 0.218 |
| Pitt Place1 | 6.8 | 85.1 | 4.3 | 1.3 | 9.3 | 0.051 |
| Pitt Place2 | 22.0 | 75.2 | 12.4 | 1.7 | 10.7 | 0.165 |
| Pitt Place3 | 1.8 | 84.7 | 6.9 | 0.7 | 7.7 | 0.081 |
| Curt Cope4 | 38.2 | 79.0 | 7.4 | 2.5 | 11.1 | 0.094 |
| Curt Cope5 | 44.7 | 77.1 | 11.8 | 2.7 | 8.4 | 0.153 |
| Curt Cope6 | 24.0 | 90.1 | 8.1 | 1.6 | 0.2 | 0.090 |

Repeated measurements at one location were conducted to determine the absolute error for each component. The error of component determination was calculated using a standard statistical equation (i.e., standard deviation multiplied by the Student's coefficient using degrees of freedom equal to the number of measurements minus one at a confidence level of 0.95) during data processing of this series of measurements. The errors received are presented in the Table 5 header.

Comparison of TNM Measurements with Other Methods

Field measurements of soil moisture and chemical composition were conducted by conventional methods, to compare the results obtained from these conventional methods with the TNM methods described herein. Moisture measurements were performed using two techniques: time domain reflectometry (TDR) with the HydroSense™ II Handheld Soil Moisture Sensor (HS2P), and a nuclear method using the Model 3440 Moisture Density Gauge from Troxler™ Electronic Laboratories, Inc. The TDR instrument used 4-inch (˜10 cm) rods, and the nuclear source on the Troxler gauge was inserted into the soil to a depth of 10 cm. Therefore, the moisture measurement results obtained by each of these conventional methods represent the average moisture content of the upper ˜10 cm layer of the soil.

Soil samples for chemical analysis were collected from cores approximately 5 cm in diameter and 30-40 cm in length, with these soil samples obtained from each of the locations where the TNM measurements, described herein, were performed. Each core was segmented into 5 cm increments. The samples were dried, grounded and sieved, with several subsamples weighing approximately 0.2 g being analyzed for carbon content using dry combustion with a TruSpec™ CN analyzer (LECO Corp., Saint Joseph, MI). The chemical analysis results showed an exponential decrease in carbon content with depth. The average carbon content in the upper 10 cm layer at each site was calculated by averaging the values from the 0-5 cm and 5-10 cm core segments.

In general, the silicon content in the soil does not vary significantly with depth, down to a soil depth of 50-100 cm. Therefore, for silicon analysis, the dried soil samples from the 0-5 cm and 5-10 cm layers were combined, thoroughly mixed, and three subsamples were taken for analysis. These subsamples were digested using a mixture of hydrofluoric acid and concentrated nitric acid, heated in Teflon tubes with the aid of microwave radiation. This process allowed for complete dissolution of the soil matrix. Silicon concentrations were then measured using an Inductively Coupled Plasma Mass Spectrometer (ICP-MS). The resulting values were attributed to the average silicon content in the upper 10 cm of the soil profile.

Since the results obtained by the TNM method (as another soil neutron-gamma analysis) may be attributed to the elemental content in the upper 10 cm layer of the soil (Kavetskiy et al., 2017), comparisons between the TNM-derived values and those obtained from the independent measurements of moisture, carbon (C), and silicon (Si) content are valid. The comparison of the results obtained by the conventional methods and the TNM methods was performed using statistical analyses, specifically the Bland-Altman plot and Deming regression. The results of the comparison are presented in Table 6. As described herein, to confirm that two measurement methods yield equivalent results, the following conditions should be met:
The mean difference between methods is close to zero;
The distribution of differences follows normality (as verified by the Jarque-Bera test (“JB test”)-should be less than the critical value of 2.72);
The p-values from both the slope and means tests are greater than 0.05;
The 0 lies within the confidence intervals of the test parameters.

As shown in Table 6, the calculated statistical values in all cases meet the conditions outlined above. Therefore, it may be concluded that the statistical analysis supports the validity of the TNM method. The measurements of soil moisture, carbon, and silicon content obtained using TNM measurements, disclosed herein, are comparable to those obtained using conventional analytical methods.

TABLE 6
Results of statistical analysis comparison of measurement moisture,
silicon and carbon content received by TNM and traditional methods

| | | | Deming regression | | | | | | | |
|---|---|---|---|---|---|---|---|---|---|---|
| | | | Slope test | | | Means test | | | | |
| | | | | Confidence | | | Confidence | | | |
| | | | | interval of test | | | interval of test | | Bland-Altman | |
| | Method | Method | p- | parameters | | p- | parameters | | Mean, | JB |
| Element | 1 | 2 | value | LCL | UCL | value | LCL | UCL | wt % | test |
| Mo | TNM | TDR | 0.48 | −0.20 | 0.31 | 0.50 | −1.75 | 3.10 | 0.93 | 0.95 |
| Mo | TNM | Nucl. | 0.41 | −0.17 | 0.35 | 0.50 | −1.75 | 3.10 | 0.12 | 1.22 |
| Mo | Nucl. | TDR | 0.90 | −0.24 | 0.22 | 0.11 | −3.50 | 0.70 | −1.40 | 0.48 |
| C | TNM | Dry | 0.30 | −0.10 | 0.24 | 0.09 | −0.05 | 0.31 | 0.13 | 0.30 |
| | | comb. | | | | | | | | |
| Si | TNM | Chem. | 0.31 | −0.28 | 0.11 | 0.10 | −0.47 | 2.55 | −1.04 | 1.20 |
| | | Analys | | | | | | | | |

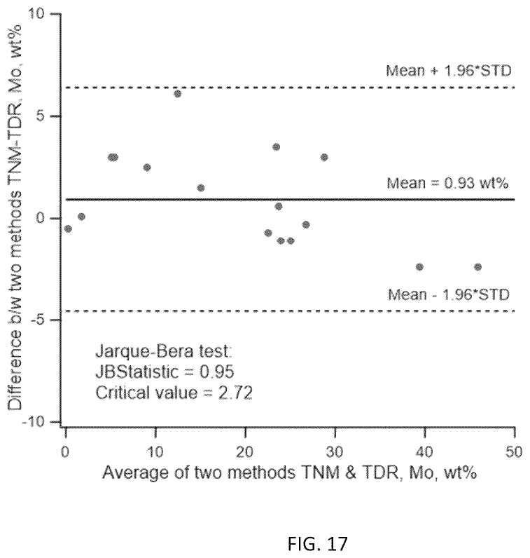

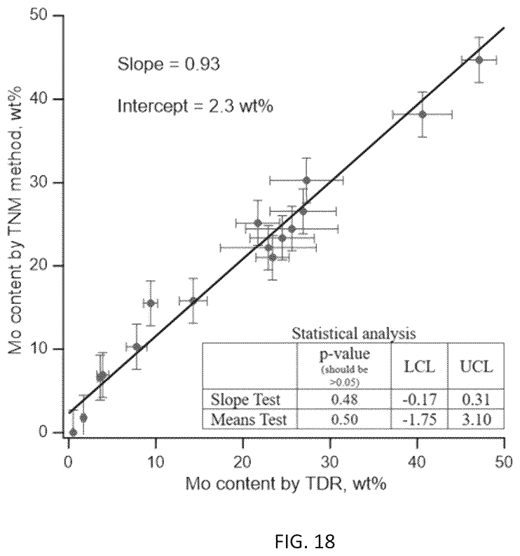

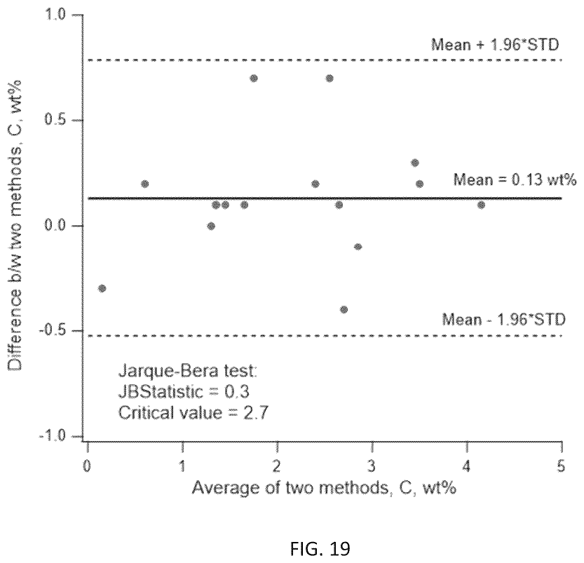

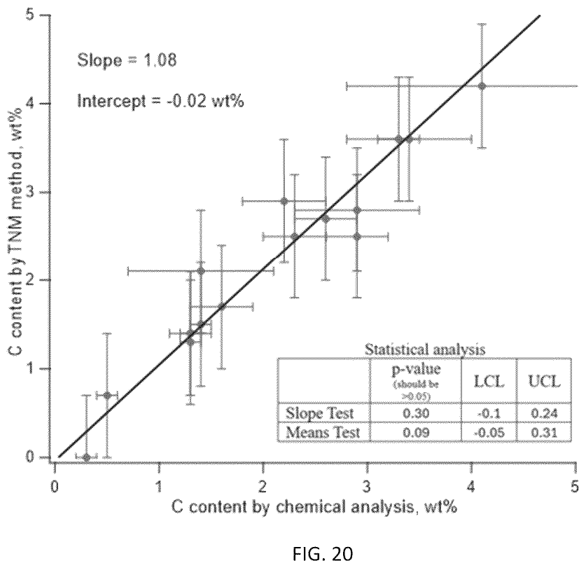

For reference, the Bland-Altman plots and Deming regression analyses are shown in FIGS. 17 to 20. As illustrated in these plots, the range of differences is narrow relative to the mean values (Bland-Altman), and the slopes of the Deming regression lines are close to 1 and the intercepts are close to 0. These observations provide additional evidence of strong agreement between the TNM method and conventional measurement techniques.

### Comparison of

$$\frac{Soil {Al}_{2} {O}_{3}}{Soil {SiO}_{2}}$$

### Ratio and Soil Texture as Measured by TNM and Reference Data

The calculated

$$\frac{Soil {Al}_{2} {O}_{3}}{Soil {SiO}_{2}}$$

ratios, obtained from measuring SiO₂ and Al₂O₃ content in soils by TNM, are shown in Table 5. These calculated ratios were compared with

$$\frac{Soil {Al}_{2} {O}_{3}}{Soil {SiO}_{2}}$$

ratios which can be determined from reference data. The reference data for the Soil bins was derived from “Properties of Bin Soil” (Batchelor, 1984), and the

$$\frac{Soil {Al}_{2} {O}_{3}}{Soil {SiO}_{2}}$$

reference data for the field measurement locations was generated using conventional laboratory analysis techniques.

It is difficult to obtain an accurate measurement of total soil SiO₂ and Al₂O₃ using conventional laboratory analysis techniques. Instead, total soil Si was determined using laboratory analysis techniques for all soil samples and used to calculate the SiO₂ of the samples. The total Al₂O₃ was calculated using reference data for the Soil bins. There is specific data and information regarding clay minerology and soil texture classification for each soil bin. Therefore, the formulas for the soil minerals in each soil bin was used to determine the amount of Al₂O₃ in the soil contained in each bin. Although some of the soil minerals may be found in trace amounts, the larger soil mineral components were determined; therefore, the actual Al₂O₃ content in the soil bins may be estimated to a reasonable level of accuracy. Regarding the laboratory analysis of the soil measured in the fields, because the Applicants found it difficult to obtain accurate total soil Al content using conventional laboratory techniques, knowledge of the clay minerology of the soils in the fields was also used to estimate, with a reasonable amount of accuracy, the total content of Al₂O₃ in the field soil samples. The comparison of

$$\frac{Soil {Al}_{2} {O}_{3}}{Soil {SiO}_{2}}$$

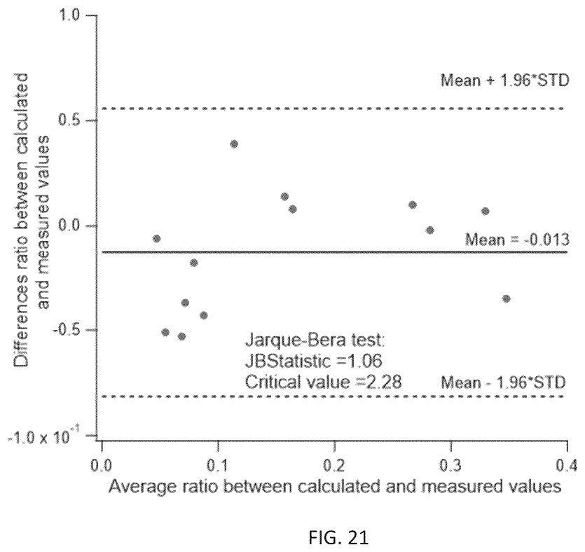

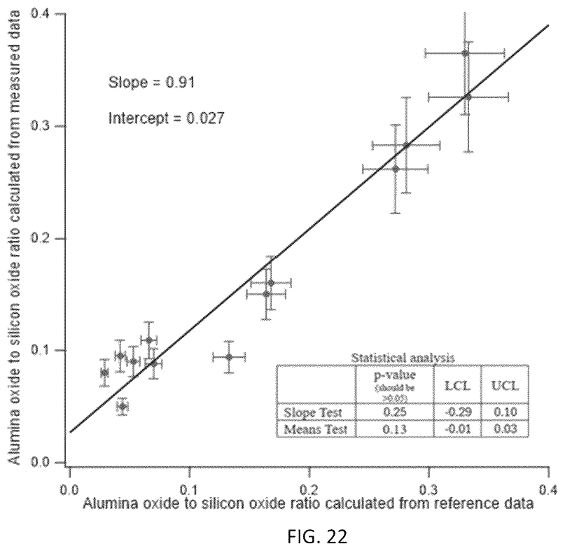

received from the reference sources (or laboratory analysis, as applicable) and from TNM measurements was conducted using Bland-Altman plots and Deming regression. As can be seen from the data represented in FIGS. 21 and 22, it may be observed that the results of both methods are comparable, which serves as evidence that using TNM measurements to calculate the

$$\frac{Soil {Al}_{2} {O}_{3}}{Soil {SiO}_{2}}$$

ratio is a viable method.

The methodology of determination of soil texture based on the

$$\frac{Soil {Al}_{2} {O}_{3}}{Soil {SiO}_{2}}$$

ratio, as calculated from TNM measurements and using the contour lines in the plot of FIG. 5 may, in some instances, provide an exact soil texture classification. In other instances, the determination of soil texture based on the

$$\frac{Soil {Al}_{2} {O}_{3}}{Soil {SiO}_{2}}$$

ratio may provide an estimation of the soil texture classification, with two or three neighboring soil texture classes identified for the soil being tested. The comparison of soil texture classifications by using the TNM methods described herein, and reference data, is provided Table 7 below. As will be appreciated, in nearly all cases, one soil texture classification obtained from TNM measurements of the

$$\frac{Soil {Al}_{2} {O}_{3}}{Soil {SiO}_{2}}$$

ratio coincided with the soil texture classification provided in the reference data. In practice, it is useful to classify a soil texture into one of three main types: Sand, Loam, or Clay. The methods disclosed herein, utilizing INS gamma spectra obtained by a TNM system, a PFTNA system, or any other system may provide, in some embodiments, a clear identification of the soil texture type of a soil. Advantageously, this in-situ method may be effectively used in agriculture as an alternative to labor-intensive and time-consuming soil sampling and laboratory analysis

TABLE 7
Comparison of Soil texture classification for soil bins and
fields based on reference data and based on TNM measurements

| Bin# or soil | Soil texture | |
|---|---|---|
| spot name | Reference data | Measurement data |
| Indoor bin | Sandy loam | Sandy loam, Sandy clay loam |
| Bin-3a | Clay | Clay |
| Bin-3b | Clay | Clay |
| Bin-4 | Sandy loam | Sandy loam, Sandy clay loam |
| Bin-5 | Clay | Clay |
| Bin-7 | Silty clay | Silty clay, Clay |
| Bin-8 | Clay | Clay |
| Bin-9a | Clay | Clay |
| Bin-9b | Silty clay | Clay |
| Lab Field | Loamy sand | Sandy loam, Sandy clay loam |
| PittPlace1 | Loamy sand | Sandy loam |
| PittPlace2 | Clay loam | Clay Loam, Loam, Silty Loam |
| PittPlace3 | Sand | Sandy loam |
| CurtCope4 | Sandy clay loam | Sandy clay loam, Sandy loam |
| CurtCope5 | Clay loam | Sandy clay loam, loam, silty loam |
| CurtCope6 | Sandy loam | Sandy loam |

### Practical Applications

Regarding practical application of determining a grouping of soil texture classes of a given soil, two of the most prevalent soil characteristics that may be shared by a grouping of soil texture classifications are: 1) water holding capacity and 2) cation exchange capacity (CEC). Water holding capacity drives how soil water moves through soil and will therefore impact erosion and runoff water quality. It also changes how plants can retrieve water from soil for growth, so that it impacts factors such as drought tolerance and irrigation rates and timing. The CEC of a soil directly impacts the ability of the soil to hold nutrients that are bioavailable to the plant, so it also directly impacts soil fertility and thus impacts fertilizer application rates and nutrient use efficiency. This is a primary soil function that drives precision fertilizer application effectiveness. The same nutrients impacted by CEC levels that are important for plant growth are also important for microbes in soil, and microbes drive soil nutrient transformation functions. Since microbial activity drives nutrient availability transformations in soil, this in turn drives the whole complicated soil/plant interactions the determine crop productivity.

Outside the realm of agriculture, the soil texture is a primary consideration for planning construction site work. Changing the level of sand and clay in soil will impact the ability of a soil to be packed. Also, when a construction site work may be performed is dependent on how wet the soil is and how quickly it will dry out, which are soil characteristics that are directly related to soil water holding capacity.

### Additional Ratio Measurements for Soil Texture Class Identification

In addition to, or as an alternative to, using the measured Al₂O₃:SiO₂ ratio of soil in a given field to identify the soil texture class of that soil, the Applicants hypothesize that other soil oxide or compound ratios may be measured and used to identify soil texture classes. In some embodiments, other soil element ratios may be used in combination with the measured Al₂O₃:SiO₂ ratio to help reduce the overlap between soil texture classes, thereby allowing for a more precise identification of the soil texture class of a given soil. For example, in particular there are other elements incorporated in the minerals that make up the structure of clay that may help distinguish clay from sand and silt. Below is a discussion of differences in clay types that illustrates the different elements in clay that may be useful for determining soil texture.

Clay minerals may be broadly grouped into a classification of either a 1:1 clay or a 2:1 clay. The 1:1 clay minerals (basic kaolin mineral) have a structure comprising of layers of a single tetrahedral sheet and a single octahedral sheet. Such 1:1 clay minerals include kaolinite, dickite, nacrite, and halloysite. The most common 1:1 clay in agriculture soils is kaolinite. The structural formula for kaolinite is Al₄Si₄O₁₀(OH)₈ and the theoretical chemical composition is SiO₂ (46.54%), Al₂O₃ (39.50%), and H₂O (13.96%). The 2:1 clay minerals (Smectite minerals) consist of an octahedral sheet sandwiched between two tetrahedral sheets. The structural formula for smectite is (OH)₄Si₈Al₄O₂₀·NH₂O (interlayer) and the theoretical chemical composition, without the interlayer material, is:SiO₂ (66.7%), Al₂O₃ (28.3%), and H₂O (5%). However, in smectites, there is considerable substitution in the octahedral sheet and some in the tetrahedral sheet. In the tetrahedral sheet, there is substitution of aluminum for silicon in amounts of up to 15% (SSURGO Database, 1993, 2014; (Batchelor, J. A., Jr. (1984); Properties of Bin Soils at the National Tillage Machine Laboratory, Pub. 218. Auburn, AL: USDA-ARS National Soil Dynamics Laboratory (“Batchelor, 1984”)). In the octahedral sheet, aluminum may be substituted by magnesium and iron. If the octahedral positions are mainly filled by aluminum, the smectite mineral is beidellite; if filled by magnesium, the mineral is saponite; and if filled by iron, the mineral is nontronite. The most common smectite mineral is calcium montmorillonite, which means that the layer charge deficiency is balanced by the interlayer of the calcium cation and water.

Another factor that may be helpful in determining soil texture through measurements of the elemental content of the soil, is that the type of clay present is typically consistent across a geographical region. While the amount of clay present across a geographical region may vary, the type of clay present does not tend to vary. Thus, in some embodiments, it may be possible to identify the ranges of Al content that would be expected to make up a clay component based on the geographic region of the soil to be analyzed. The 1:1 clays are very consistent as to the percentage Al, and the most common 2:1 clay in agriculture soil is calcium montmorillonite. The other major smectite minerals contain Na, Mg, and Fe which may also be measured using the mobile gamma measurement techniques described above. For example, the TNM methods may be used for calculating elemental ratios in the soil of Na, Mg, Fe and other elements, so long as the element under analysis has a clear, characteristic peak in a gamma spectrum produced by inelastic neutron scattering (INS). Knowledge of the clay type that is expected in a given geographical region may be used to provide the percentage of Al, Na, Mg and/or Fe in the clay to be used in the ratio calculations described above.

As a general rule, the more weathered a soil is, the more clay it contains. The use of element ratios to distinguish changes in soil has been used in the scientific literature by geologist to develop indexes to measure weathering based on different chemical ratios in soil; for example, see: Heidari et al, “Geochemical indices as efficient tools for assessing the soil weathering status in relation to soil taxonomic classes”, Catena 208 (2022) 105716. An example, not intended to be limiting, of a potentially useful ratio is the measurement of SiO₂:Fe₂O₃ contained in the soil, as measured by the mobile gamma measurement techniques described herein.

### Natural Background Radiation Measurements for Soil Texture Class Identification

Another possible approach, which may be used in the alternative or in combination with the soil chemical composition ratios described herein, is to measure the background radiation emitted by certain radioisotopes that are naturally present in the soil.

In one aspect, the Applicants hypothesize that measurements of the natural soil background gamma radiation, as produced by radioactive elements naturally present in the soil, may be used to estimate or identify the soil texture classification of a soil. For example, not intended to be limiting, soil may contain one or more of the following primordial radioisotopes:
Potassium: 40K decays to produce 40Ar
Thorium: ²³²Th decays to produce ²⁰⁸Tl
Uranium: ²³⁸U decays to produce ²¹⁴Bi

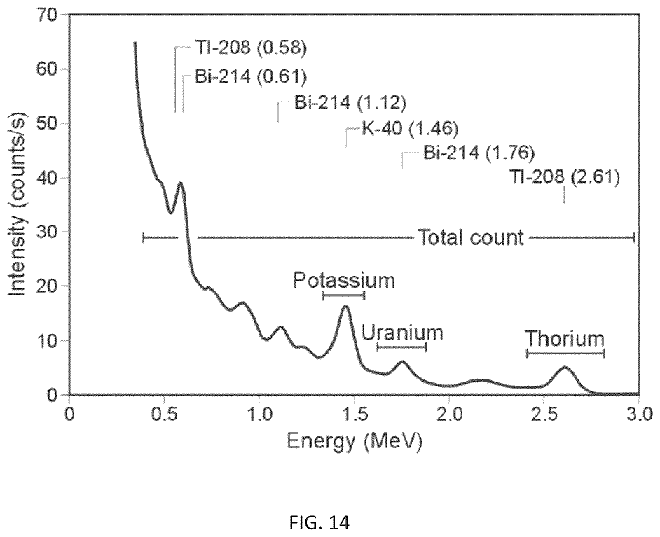

The gamma radiation produced by the decay of Potassium-40, Thallium-208 and Bismuth-214 results in characteristic gamma peaks, as shown for example in the gamma spectrum of FIG. 14 (FIG. 14 excerpted from B. Minty, 1997. Fundamentals of airborne gamma-ray spectrometry. AGSO journal of Australian geology & geophysics 17 (2): 39-50). The different mineral components of the silt, sand and clay of a given soil will contain different amounts of the above-noted primordial radioisotopes. Therefore, the measurement of the natural background gamma radiation of a soil using mobile gamma analysis may be used to correlate such measurements to different soil texture classes.

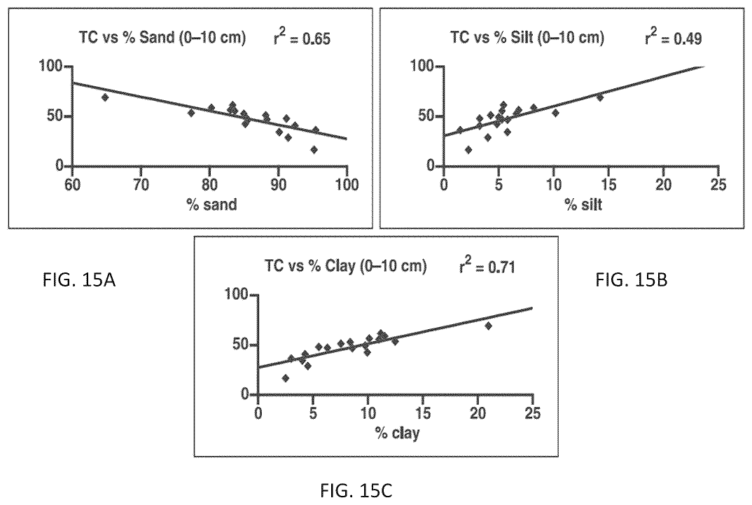

In one example, the technique of determining the soil texture classification of a soil using measurement of the natural gamma radiation of a soil involves correlating the total counts in the gamma spectra, representing the total background gamma radiation of the soil emitted by all radioisotopes present in the soil, to the varying content of sand, silt and clay contained in the soil. For example, see FIGS. 15A to 15C, which provides data from a study showing a correlation between the total counts and the percentage of sand, silt and clay, respectively (FIGS. 15A to 15C excerpted from: M. J. Taylor, K. Smettem, G. Pracilio and W. Verboom, 2002, “Relationships between soil properties and high-resolution radiometrics, central eastern Wheatbelt, Western Australia” Exploration Geophysics 33:95-102. ISBN 9076998213). In this data, it appears that higher total counts are correlated with lower sand content and higher silt and clay content; whereas, lower total counts are correlated with higher sand content and lower silt and clay content.

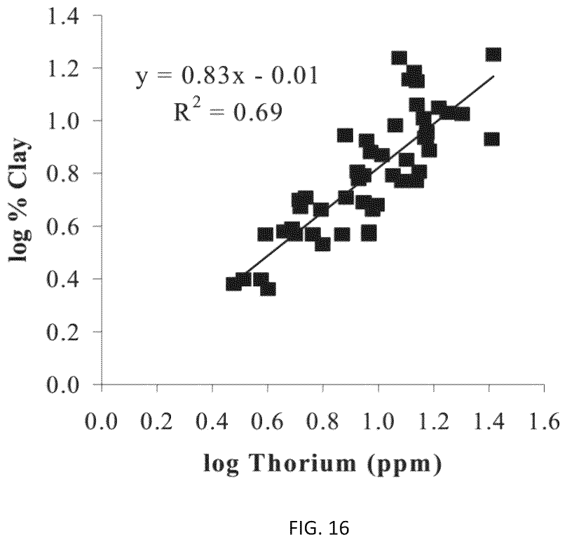

In another example, a measurement of the volumetric concentration of one or more of the primordial radioisotopes, mentioned above, may be correlated with the percentage of silt, sand, and/or clay in a soil under analysis. For example, as shown in the plot at FIG. 16, in a study it was demonstrated there may be a linear correlation between the logarithmic content of Thorium (ppm) and the logarithmic percentage of clay in a soil sample (FIG. 16 excerpted from G. Pracilio, M. L. Adams and K. R. J. Smettem. 2003, “Use of airborne gamma radioimetric data for soil property and crop biomass assessment” Proceedings of the 4th European Conference: pp. 551-557. Ed: J. V. Stafford, A. Werner, Berlin, Germany. Wageningen Academic Publishers).

The measurement of thorium, potassium and/or uranium, either alone or in combination, that is naturally present in a soil, may be utilized, either alone or in combination with the soil elemental chemistry ratio techniques described herein, to identify the soil texture class of a soil. Such measurements, including the TNM measurements and the natural background radiation measurements, may be accomplished simultaneously utilizing the same mobile gamma measurement apparatus.

What is claimed is:

1. A method for identifying a soil texture class of a soil, the method comprising:
acquiring an inelastic neutron scattering (INS) gamma spectrum of the soil,
calculating at least one ratio of a mass fraction of a first oxide to a mass fraction of a second oxide present in the soil, wherein the mass fraction of each of the first and second oxides is determined from calculating a contribution to a characteristic peak in the gamma spectrum of the soil by each oxide of the first and second oxides, and
identifying one or more soil texture classes of the soil by identifying a contour line of a contour plot that corresponds to the calculated at least one ratio, wherein the contour line correlates the calculated at least one ratio to one or more soil texture classes.

2. The method of claim 1 wherein the step of calculating the at least one ratio of the mass fractions of the first and second oxides further comprises performing a deconvolution procedure on the acquired gamma spectrum, wherein the deconvolution procedure applies a least squares method for determining the mass fraction of each of the first and second oxides.

3. The method of claim 2, wherein the deconvolution procedure is modified to account for radiation attenuation by components in the soil.

4. The method of claim 1 wherein the first oxide is SiO₂.

5. The method of claim 4 wherein the second oxide is selected from a group comprising: Al₂O₃, Fe₂O₃.

6. The method of claim 1, wherein the INS gamma spectrum of the soil is acquired using a Tagged Neutron Method (TNM) system.

7. The method of claim 6, wherein the soil is in a field and wherein the step of acquiring the INS gamma spectrum of the soil further comprises moving the TNM system across the field in a point sampling mode to obtain a plurality of INS gamma spectra of the soil.

8. The method of claim 7 wherein the method further includes the step of acquiring a geographic coordinates for a position on the field where each INS gamma spectrum of the plurality of INS gamma spectra is obtained.

9. The method of claim 1 wherein the INS gamma spectrum of the soil is obtained using a Pulsed Fast Thermal Neutron Analysis (PFTNA) system.

10. The method of claim 9, wherein the soil is in a field and wherein the step of acquiring the INS gamma spectrum of the soil further comprises moving the PFTNA system across the field in a scanning mode to obtain a plurality of INS gamma spectra of the soil.

11. The method of claim 10, wherein the method further includes the step of acquiring a geographic coordinates for a position on the field where each INS gamma spectrum of the plurality of INS gamma spectra is obtained.

12. The method of claim 1 wherein the step of calculating the at least one ratio of the mass fraction of the first oxide to the mass fraction of the second oxide present in the soil comprises calculating a first ratio of the mass fraction of the first oxide to the mass fraction of the second oxide present in the soil, and calculating a second ratio of a mass fraction of a third oxide to a mass fraction of a fourth oxide present in the soil, and
wherein the step of identifying one or more soil texture classes of the soil comprises:
identifying a contour line of a first contour plot that corresponds to the calculated first ratio, the identified contour line of the first contour plot correlating the first calculated ratio to a first grouping of one or more soil texture classes,
identifying a contour line of a second contour plot that corresponds to the calculated second ratio, the identified contour line of the second contour plot correlating the second calculated ratio to a second grouping of one or more soil texture classes,
identifying an overlap between the first and second groupings of one or more soil texture classes to determine the soil texture class of the soil.

13. The method of claim 12 wherein the second and fourth oxides are each SiO₂.

14. A system for identifying a soil texture class of a soil, the system comprising:
a neutron generator assembly for generating neutrons and directing the generated neutrons into the soil,
a gamma detector assembly for detecting the gamma radiation emitted by the soil,
a radiation shielding positioned between the neutron generator assembly and the gamma detector assembly,
a processor in communication with the gamma detector assembly, the processor configured to:
acquire an INS gamma spectrum from the gamma radiation detected by the gamma detector assembly,
calculate a mass fraction of each of at least a first and second oxide present in the soil, each mass fraction of each oxide based on a net peak area of a characteristic peak of each oxide obtained from the acquired gamma spectrum,
calculate at least one ratio of the mass fractions of the at least first and second oxides present in the soil, and
record the acquired gamma spectrum and the calculated at least one ratio to a memory.

15. The system of claim 14 wherein the neutron generator assembly also generates alpha particles and wherein the system further comprises an alpha detector assembly.

16. The system of claim 15 wherein the gamma detector assembly is positioned spaced apart from, and laterally of, the neutron generator.

17. The system of claim 15 wherein the alpha detector assembly and the soil are positioned on opposite sides of the neutron generator assembly.

18. The system of claim 14 wherein the radiation shielding comprises one or more of the following: lead, borated polyethylene, borated-lead polyethylene.

19. The system of claim 14 wherein the system is mounted to a mobile cart and wherein the system further comprises a global positioning system (GPS) and wherein the processor is configured to record a plurality of gamma spectra of the soil and to save a geographic coordinate of the location of each acquired gamma spectrum of the plurality of gamma spectra to the memory.

20. The system of claim 14 wherein the processor is additionally configured to identify one or more soil texture classes of the soil by identifying a contour line of a contour plot that corresponds to the calculated at least one ratio, wherein the contour plot correlates the calculated at least one ratio to one or more soil texture classes.

21. The system of claim 14 wherein the processor is additionally configured to perform a deconvolution procedure on the acquired gamma spectrum, the deconvolution procedure comprising applying a least squares method for determining the mass faction of each of the at least first and second oxides present in the soil.

22. The system of claim 21 wherein the deconvolution procedure is modified to account for radiation attenuation by components in the soil.
